(19) United States
(12) Patent Application Publication
(10) Pub. No.: US 2024/0299776 A1
(43) Pub. Date: Sep. 12, 2024

(54) METHOD AND TREATMENT OF THE EYE WITH ULTRASOUND

(71) Applicant: EyeSonix Inc., Los Angeles, CA (US)

(72) Inventors: Donald N. SCHWARTZ, Los Angeles, CA (US); Eric SCHULTZ, Los Angeles, CA (US)

(73) Assignee: EyeSonix Inc., Los Angeles, CA (US)

(21) Appl. No.: 18/608,503

(22) Filed: Mar. 18, 2024

Related U.S. Application Data
(63) Continuation of application No. PCT/US2024/019232, filed on Mar. 8, 2024.
(60) Provisional application No. 63/509,724, filed on Jun. 22, 2023.
(60) Provisional application No. 63/451,154, filed on Mar. 9, 2023.

(51) Int. Cl. A61N 7/00; A61P 27/06

(52) U.S. Cl. CPC A61N 7/00; A61P 27/06; A61N 2007/0021

(57) ABSTRACT

The present disclosure provides devices, systems and methods for treating eye disorders by delivering ultrasound energy. Specifically, methods for reducing or reversing retinal ganglion nerve layer (RNFL) thinning are provided.

### 1. CROSS REFERENCE TO RELATED APPLICATIONS

This application is a continuation application of International Application No.: PCT/US2024/019232, filed Mar. 8, 2024, which claims the benefit of and priority to U.S. Provisional Application No. 63/451,154, filed Mar. 9, 2023; and U.S. Provisional Application No. 63/509,724, filed Jun. 22, 2023, each of which is hereby incorporated by reference in its entirety.

### 2. FIELD

Disclosed herein are devices, systems and methods relating to the treatment of eye disorders or ocular conditions using low intensity ultrasonic energy.

### 3. BACKGROUND

The retinal nerve fiber layer (RNFL) plays a crucial role in visual function, serving as a conduit for transmitting visual information from the retina to the brain. Maintaining the integrity and thickness of the RNFL is essential for optimal visual health. Various diseases and conditions, such as glaucoma, optic neuritis, and certain vascular disorders, can lead to thinning of the RNFL, resulting in visual impairment and potentially irreversible damage if left untreated.

Current approaches to managing RNFL thinning primarily focus on early detection and monitoring of disease progression through imaging techniques such as optical coherence tomography (OCT). While these methods provide valuable diagnostic information, there remains a significant need for therapeutic interventions aimed at preserving or enhancing RNFL thickness.

Recent research has identified potential strategies for increasing RNFL thickness, including neuroprotective agents, growth factors, and regenerative therapies. However, many of these approaches face challenges such as limited efficacy, invasive procedures, and systemic side effects.

There exists, therefore, a need for a non-invasive, targeted method capable of safely and effectively increasing RNFL thickness to mitigate the progression of visual impairment associated with RNFL thinning. Such a method would offer significant advancements in the treatment of various ocular diseases and conditions, improving patient outcomes and quality of life.

### 4. BRIEF DESCRIPTIONS OF THE DRAWINGS

These and other features, aspects, and advantages of the present invention will become better understood with regard to the following description, and accompanying drawings, where:

FIG. 1 is an illustration of the application of an ultrasonic device to the eye. The tip of the ultrasonic device is called the sonotrode tip, i.e., horn (101) and is from where the energy emanates. The horn is placed against a conjunctiva (102), a thin layer of tissue above the sclera (110) and is aimed at the Canal of Schlemm (107), the ciliary body (103) and the trabecular meshwork (106), as described in some embodiments herein. The iris (109) and trabecular meshwork (106) form what is known as the anterior chamber angle. The primary point of contact of the horn to the conjunctiva is at a location known as the limbus (108). For anatomical reference, the horn is also placed below the cornea (104) and generally above the ciliary body (103). The ultrasonic beam and/or energy (105) represents the dispersion of energy typically found in this general design of the horn.

FIG. 2 depicts a diagram illustrating that the use of ultrasound (201) corresponds to an increased presence of several biochemicals (202) that are known to affect several cellular processes linked to neuroregeneration (203), as described in some embodiments herein.

FIG. 3 depicts a diagram showing an example of how a sensor may be used to alter parameters of the ultrasound energy system that benefits device functionality, as described in some embodiments herein.

FIG. 4 depicts a diagram showing an example of how physiologic parameters of the patient may be used to enhance patient safety by adjusting the ultrasound energy system appropriately, as described in accordance with some embodiments herein.

FIGS. 5A-5D show Heidelberg Retinal Tomography images that show information regarding a patient's treated eye, and non-treated contralateral eye before and after treatment with the device, as described in accordance with some embodiments herein. FIG. 5A shows data from the treated eye (OD) of patient 254 before treatment; FIG. 5B shows data from the contralateral eye (OS) of patient 254 before treatment; FIG. 5C provides data from the treated eye (OD) of patient 254 after treatment; and FIG. 5D provides data from the contralateral eye (OS) of patient 254 after treatment.

FIGS. 6A-6E show example output files from a vision field analyzer that show the same eye of a patient before and after a single treatment with the device, as described in accordance with some embodiments herein. FIGS. 6A and 6B show ultrasound treatment increased a patient's vision field index (VFI) from 61% before treatment (FIG. 6A) to 74% after treatment (FIG. 6B). The mean deviation of vision loss (MD) decreased from a loss of 15.63 dB (FIG. 6A) to 10.97 dB (FIG. 6B). FIGS. 6C, 6D and 6E show ultrasound treatment increased VFI in a patient (Patient 262) from 79% when Patient 262 was on medications to 84% when ultrasound treatment was used in combination with medication. ultrasound treatment recovered Patient 262's paracentral vision (arrow in FIG. 6E).

FIGS. 7A-7H illustrate that an exemplary porcine eye treated with the device had increased biomarkers expression in the trabecular meshwork. FIG. 7B shows a greater presence of TNF-alpha in the trabecular meshwork than the control eye (FIG. 7A) which did not receive treatment. FIG. 7C shows ultrasound treatment increased TNF-alpha expression as quantified by ELISA assay. FIG. 7D shows the effect of ultrasound on TNF-alpha expression was comparable to the impact of laser treatment on TNF-alpha. FIG. 7F shows ultrasound did not change expression level of IL-1 beta as compared to the control (FIG. 7E). ELISA assay quantification showed ultrasound had no significant impact on IL-1 beta expression. FIG. 7H shows laser treatment increased IL-1 beta expression.

FIGS. 8A-8C show exploded (FIG. 8A), right planar (FIG. 8B), and right planar cross-sectional (FIG. 8C) views of the device, as described in accordance with some embodiments herein.

FIGS. 9A-9H provide data showing that a bovine eye treated with ultrasound had increased biomarkers expression in the ciliary body. Expression of beta actin was used as internal controls. FIGS. 9A and 9B show a greater presence of mTOR in the ciliary body of the bovine eye treated with ultrasound compared to the untreated eye. FIGS. 9C and 9D show no significant difference of mTOR in the trabecular meshwork in the eye with or without ultrasound treatment. FIGS. 9E and 9F show increased mTOR in aqueous humor after ultrasound treatment. FIGS. 9G and 9H show significant reduction of mTOR in the retina after ultrasound treatment.

### 5. SUMMARY

The present disclosure provides methods and devices for treating eye disorders. The device is designed to deliver ultrasound energy that is divergent and has low intensity and low frequency to the limbus of the subject's eye such that the ultrasonic energy is propagated throughout the eye. The methods can be used repeatedly to achieve additive effects. Effective treatment schedule can stimulate cell growth in the retina nerve fiber layer (RNFL) in the subject, increase RNFL growth, and/or prevent or reduce RNFL thinning due to the eye disorder or natural decrease with age.

In one aspect, described herein are methods of treating a subject suffering from or at risk of an eye disorder, the method comprising:
providing ultrasonic energy to the limbus of an eye at an effective treatment schedule using an ultrasonic device that emits ultrasonic energy of between 0.01-5 watts of power,
wherein the ultrasonic energy is concentrated at a distance between 0.01-10 mm from the tip of the sonotrode of the ultrasonic device,
wherein the ultrasonic energy propagates throughout the eye,
wherein the effective treatment schedule is sufficient to stimulate cell growth in the retina nerve fiber layer (RNFL) in the subject.

In one aspect, described herein are methods of increasing thickness of retina nerve fiber layer (RNFL) in a subject, the method comprising:
providing ultrasonic energy to the limbus of an eye at an effective treatment schedule using an ultrasonic device that emits ultrasonic energy of between 0.01-5 watts of power,
wherein the ultrasonic energy is concentrated at a distance between 0.01-10 mm from the tip of the sonotrode of the ultrasonic device,
wherein the ultrasonic energy propagates throughout the eye,
wherein the effective treatment schedule is sufficient to modulate one or more biomarkers involved in promoting cell growth in the retina nerve fiber layer (RNFL), thereby increasing thickness of the RNFL.

In accordance with some embodiments, the one or more biomarkers are selected from mammalian target of rapamycin (mTOR), wingless-related integration site (WNT), nerve growth factor (NGF), glial cell line-derived neurotrophic factor (GDNF), brain-derived neurotrophic factor (BDNF), interleukin 1 (IL-1), interleukin 6 (IL-6), interleukin 8 (IL-8), tumor necrosis factor alpha (TNF-α), matrix metalloproteinase 3 (MMP-3), Ak strain transforming 1 (Akt1), paired box 6 (Pax6), B-cell leukemia/lymphoma 2 (Bcl-2), ciliary neurotrophic factor (CNTF), heat shock protein 70 (HSP70), and brain-specific homeobox/POU domain protein 3 (Brn3a).

In accordance with any one of the embodiments, the one or more biomarkers are selected from brain-derived neurotrophic factor (BDNF), nerve growth factor (NGF), glial cell line-derived neurotrophic factor (GDNF), matrix metalloproteinase 3 (MMP-3), mammalian target of rapamycin (mTOR), tumor necrosis factor alpha (TNF-α), and combinations thereof.

In accordance with any one of the embodiments, the effective treatment schedule is sufficient to increases or decrease expression level of biomarkers selected from neurotrophin 3, CNTF, HSP70, or any combination thereof.

In accordance with any one of the embodiments, wherein the effective treatment schedule is sufficient to increase or decrease gene expression level of mTOR, WNT, NGF, GDNF, BDNF, IL-1, IL-6, IL-8, TNF-alpha, Akt1, Pax6, Bcl-2, and Brn3a in retinal ganglion cells (RGC).

In accordance with any one of the embodiments, the effective treatment schedule is sufficient to increase or decrease protein expression level of mTOR, WNT, NGF, GDNF, BDNF, IL-1, IL-6, IL-8, TNF-alpha, Akt1, Pax6, Bcl-2, and Brn3a in retinal ganglion cells (RGC).

In accordance with any one of the embodiments, the effective treatment schedule is sufficient to increase axonal growth in the RNFL or thickness of the RNFL.

In accordance with any one of the embodiments, the effective treatment schedule is sufficient to increase RNFL thickness by at least 1%, 2%, 3%, 4%, 5%, 10%, 15%, 20%, 30%, 40%, 50%, 100%, 150%, 200%, 300%, 400% or more as compared to baseline.

In accordance with some embodiments, the RNFL thickness is measured by Heidelberg Retinal Tomography (HRT) or optical coherence tomography (OCT).

In accordance with any one of the embodiments, the effective treatment schedule is sufficient to increase the RNFL thickness in both treated and untreated eyes.

In accordance with any one of the embodiments, the ultrasound energy is applied to one eye or both eyes.

In accordance with any one of the embodiments, the effective treatment schedule is sufficient to decrease a cup to disc ratio of the eye, optionally wherein the ratio is measured by Optical coherence tomography (OCT) or Heidelberg Retinal Tomography (HRT).

In accordance with any one of the embodiments, the effective treatment schedule is sufficient to reduce intraocular pressure (IOP) in the subject's eye.

In accordance with some embodiments, the effective treatment schedule is sufficient to reduce the IOP by at least 20%, at least 25%, at least 30%, at least 35%, at least 40%, at least 45%, or at least 50% as compared to baseline.

In accordance with some embodiments, the effective treatment schedule is sufficient to reduce the IOP to the level of a healthy eye or lower.

In accordance with any one of the embodiments, the effective treatment schedule is sufficient to increase vision field of the eye.

In accordance with any one of the embodiments, the effective treatment schedule comprises two or more sessions of providing ultrasonic energy to the limbus of an eye.

In accordance with some embodiments, the effective treatment schedule comprises two sessions of providing ultrasonic energy to the limbus of an eye.

In accordance with some embodiments, the two sessions are scheduled 1 month, 3 months, 6 months or 12 months apart from each other.

In accordance with any one of the embodiments, the effective treatment schedule comprises three sessions of providing ultrasonic energy to the limbus of an eye.

In accordance with any one of the embodiments, the effective treatment schedule comprises providing ultrasonic energy to the limbus of an eye every 1 month, 3 months, 6 months, or 12 months or more.

In accordance with any one of the embodiments, the effective treatment schedule comprises providing ultrasonic energy to the limbus of an eye over a period of 3 months, 6 months, 9 months or 12 months.

In accordance with any one of the embodiments, the subject has glaucoma or suspected of having glaucoma.

In accordance with any one of the embodiments, the subject has not been diagnosed with glaucoma or is not suspected of having glaucoma.

In accordance with any one of the embodiments, the subject has advanced visual disease, elevated intraocular pressure, diurnal intraocular pressure (IOP), old age, decreased central corneal thickness, disc hemorrhage, genetic mutations, large beta zone of peripapillary atrophy, or optic neuropathies.

In accordance with any one of the embodiments, the subject has elevated intraocular pressure.

In accordance with any one of the embodiments, the subject has an IOP of between about 22 mmHg to about 35 mmHg before ultrasonic treatment.

In accordance with any one of the embodiments, the subject has open-angle glaucoma (OAG) or ocular hypertension.

In accordance with any one of the embodiments, the subject has damages in optic nerve.

In accordance with any one of the embodiments, the subject does not have elevated intraocular pressure.

In accordance with any one of the embodiments, the subject has normotensive glaucoma.

In accordance with any one of the embodiments, the subject has retinal nerve fiber layer (RNFL) thinner than 80 μm, thinner than 70 μm, thinner than 60 μm, thinner than 50 μm, thinner than 40 μm or thinner than an average RNFL thickness in a healthy adult.

In accordance with some embodiments, the subject has anemia, multiple sclerosis, age-related RNFL loss, retinitis pigmentosa, anterior ischemic optic neuropathy, high myopic retinal degeneration, or macular degeneration.

In accordance with any one of the embodiments, the subject has not received alternative glaucoma treatment before the ultrasonic treatment, optionally wherein the alternative glaucoma treatment is selected from intervention surgery and pharmaceutical treatments.

In accordance with any one of the embodiments, the subject has not received alternative glaucoma treatment before the ultrasonic treatment, wherein the alternative glaucoma treatment is selected from β-blockers, carbonic anhydrase inhibitors, prostaglandin analog, a2-adrenergic agonists, parasympathomimetic drugs, and combinations thereof.

In accordance with any one of the embodiments, the subject has received one or more alternative glaucoma treatments before the ultrasonic treatment.

In accordance with some embodiments, the effective treatment schedule is sufficient to reduce need for the alternative glaucoma treatment.

### Other Steps

In accordance with any one of the embodiments, the method further comprising adjusting the temperature of a portion of the eye to a temperature of between about 41 and about 45 degrees, about 40 and about 48 degrees, or about 35 and about 55 degrees Centigrade.

In accordance with any one of the embodiments, the ultrasonic energy from the ultrasonic device has about 0.5-3.99 watts of power.

In accordance with any one of the embodiments, the ultrasonic energy from the ultrasonic device has about 2.0-3.99 watts of power.

In accordance with any one of the embodiments, the ultrasonic energy from the ultrasonic device has about 3.5 watts of power.

In accordance with any one of the embodiments, the ultrasonic energy from the ultrasonic device has less than 3 watts of power.

In accordance with any one of the embodiments, the ultrasonic energy from the ultrasonic device has an effective intensity of about 0.1 watts/cm², about 0.2 watts/cm², about 0.3 watts/cm², about 0.4 watts/cm², about 0.5 watts/cm², about 0.6 watts/cm², about 0.7 watts/cm², about 0.8 watts/cm², about 0.9 watts/cm², about 1.0 watts/cm², about 1.1 watts/cm², about 1.2 watts/cm², about 1.3 watts/cm², about 1.4 watts/cm², about 1.5 watts/cm², about 1.6 watts/cm², about 1.7 watts/cm², about 1.8 watts/cm², about 1.9 watts/cm², about 2 watts/cm², about 2 watts/cm², about 2.2 watts/cm², about 2.5 watts/cm², about 2.8 watts/cm², or about 3.0 watts/cm².

In accordance with any one of the embodiments, the ultrasonic energy concentrated at the distance from the tip of the sonotrode of the ultrasonic device is less than 4 watts of power.

In accordance with some embodiments, the ultrasonic energy concentrated at the distance from the tip of the sonotrode of the ultrasonic device is about 3 watts of power.

In accordance with any one of the embodiments, the ultrasonic energy from the ultrasonic device has about 30-60 kHz frequency.

In accordance with any one of the embodiments, the ultrasonic energy from the ultrasonic device has about 35-45 kHz frequency.

In accordance with any one of the embodiments, the ultrasonic energy from the ultrasonic device has about 45 kHz or 40 KHz frequency.

In accordance with any one of the embodiments, the ultrasonic energy is provided for between 5 seconds and 120 seconds.

In accordance with some embodiments, the ultrasonic energy is applied for about 45 seconds.

In accordance with any one of the embodiments, the ultrasonic energy is applied as 10 to 15 applications wherein the 10 to 15 applications are applied while moving the tip of the sonotrode in one direction on the limbus.

In accordance with some embodiments, the ultrasonic energy is applied as 12 applications at 12 clock hour positions on the limbus.

In accordance with some embodiments, the 10 to 15 applications are applied while moving the tip of the sonotrode in the clockwise or counterclockwise direction.

In accordance with any one of the embodiments, each of the applications lasts between 5 seconds and 120 seconds.

In accordance with any one of the embodiments, the ultrasonic intensity is concentrated at a distance between 0.01-2.00 mm, 0.01-10 mm, 0.5-5 mm, or 1-3 mm from the tip of the sonotrode of the ultrasonic device.

In accordance with any one of the embodiments, the ultrasonic intensity is concentrated at a distance about 0.5 mm, about 1 mm, about 1.5 mm, or about 2 mm from the tip of the sonotrode of the device.

In accordance with any one of the embodiments, the ultrasound energy is provided at 45 degrees with respect to a normal vector of a surface of a limbus.

In accordance with any one of the embodiments, the ultrasound energy is divergent, low intensity and low frequency ultrasonic energy.

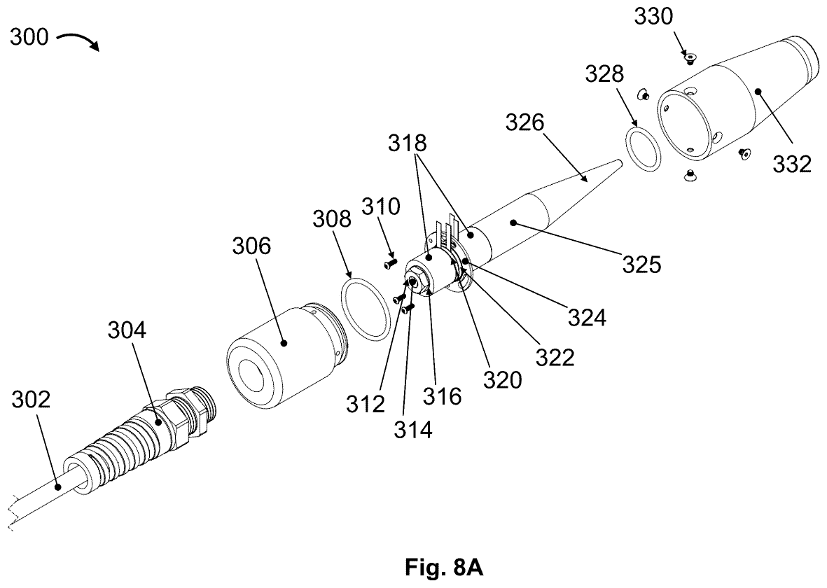

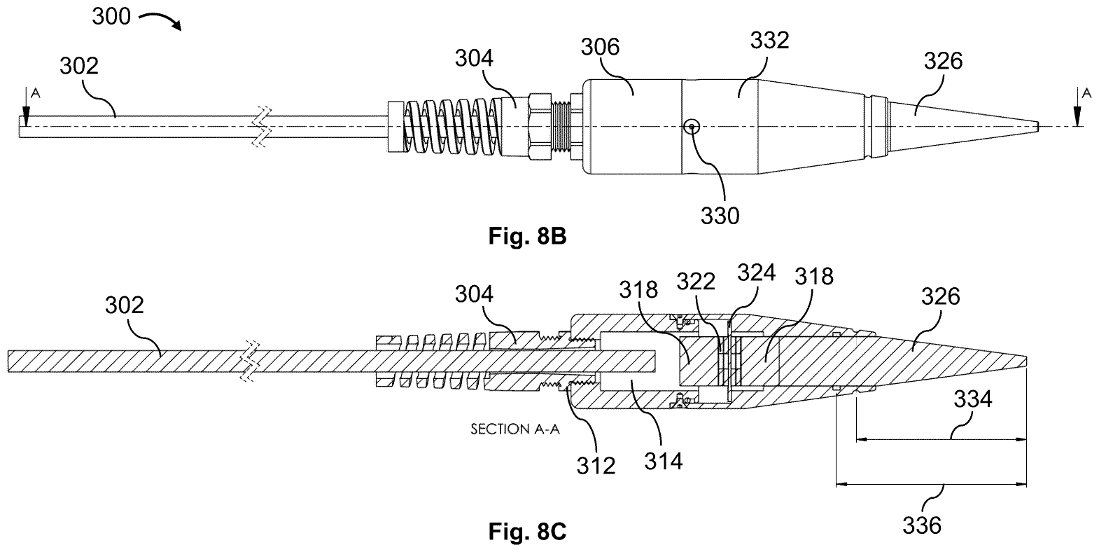

In accordance with any one of the embodiments, the ultrasonic device is as described in FIGS. 8A-8C.

In accordance with any one of the embodiments, the ultrasonic energy is provided toward the trabecular meshwork of the subject's eye.

In one aspect, described are devices for use in a treatment method, wherein the treatment method in accordance with any one of the embodiments.

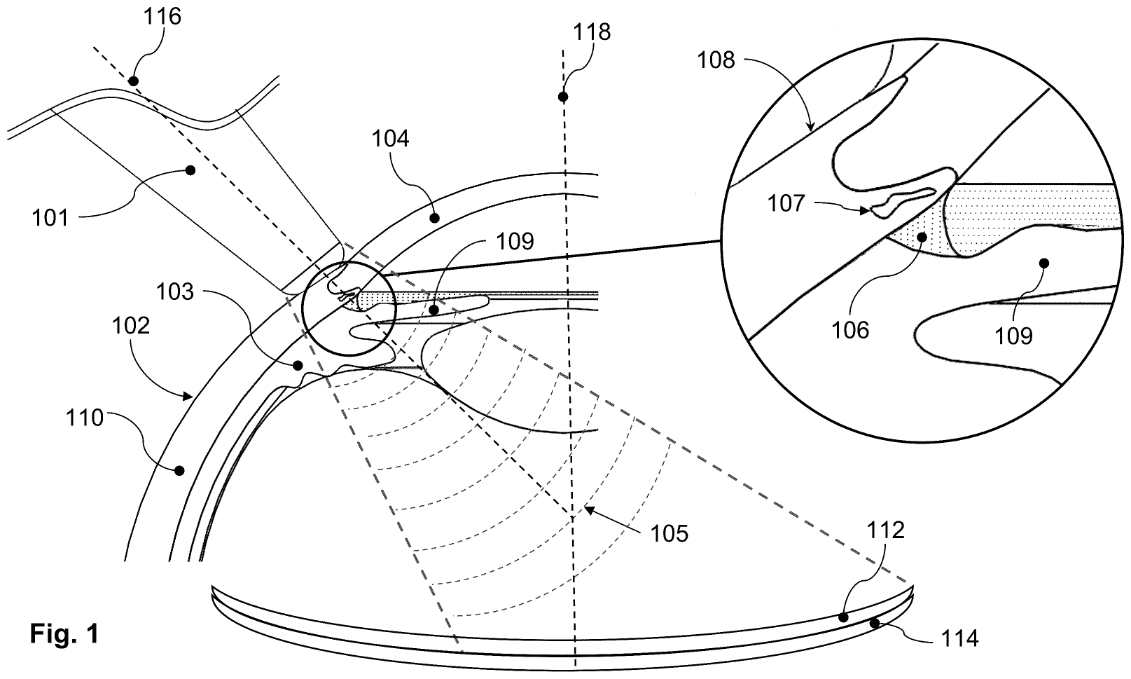

In accordance with any one of the embodiments, the movement of the sonotrode tip is between 1-10 μm. In some embodiments, the movement of the sonotrode tip is defined by the axial displacement of the sonotrode tip in either direction from an unmovable point along the sonotrode called the node. In some cases, the sonotrode tip 101 may be placed against a surface of conjunctiva overlaying the sclera 110 as shown in FIG. 1, to provide a treatment, described elsewhere herein. In some cases, the movement of the sonotrode tip 101 may comprise movement towards and away from the surface of conjunctiva 102 along an axis 116 normal to the surface of the conjunctiva 102 that the sonotrode tip 101 is placed in contact with. In some cases, the axis 116 may be at angle with respect to the optical axis of the eye 118. In some cases, the angle may comprise at least about 45 degrees of an angle. In some instances, the movement may comprise a force exerted by the sonotrode tip 101 onto the conjunctiva and sclera as the sonotrode tip 101 moves towards and away from the surface of the conjunctiva 102. In some cases, the force exerted by the sonotrode tip 101 may comprise a force exerted on the trabecular meshwork 106. The force exerted on the trabecular meshwork may dislodge a particle and/or debris to open a path through the trabecular meshwork 106.

In some cases, the treatment may be provided at one or more spatially independent or overlapping regions and/or locations on a surface of a subject's exterior ocular tissue e.g., the conjunctiva 102 overlaying the sclera 110. In some cases, the one or more treatment regions may comprise treatment as provided by contact of the device sonotrode tip 101 on the surface of the conjunctiva 102 over the sclera 110 as described elsewhere herein. In some cases, the tip may comprise an orientation and/or spatial position e.g., the angle of the sonotrode tip 101 with respect to the optical axis of the eye 118, with respect to one or more anatomical ocular tissues and/or features. In some cases, the one or more spatially independent or overlapping treatment regions may be oriented around a circular path concentric around one or more ocular features e.g., the lens or pupil of the subject's eye. In some cases, the one or more spatially independent or overlapping treatment regions may be position along a line or a curvilinear line. In some cases, the one or more independent or overlapping treatment regions may be positioned along a path with an equal or non-equal spacing between the one or more treatment regions. Treatment response to the subject may be optimized by the placement and/or location of the one or more independent and/or overlapping treatment regions. For example, a subject may have a concentrated spatial anatomical abnormality (e.g., a plug in the trabecular network or a spatially discrete thinning of the retinal fiber layer) that may be targeted by spatial independent treatment region as provided by the devices, described elsewhere herein.

In accordance with any one of the embodiments, the ultrasonic device delivers between about 0.5-2.5 watts of acoustic power to the trabecular meshwork and the ciliary body of the eye. In some embodiments, ultrasonic device delivers between about 1.5-2 watts of acoustic power to the trabecular meshwork and the ciliary body of the eye.

In accordance with any one of the embodiments, the ultrasonic device delivers about 2 watts of acoustic power to the trabecular meshwork and the ciliary body of the eye.

In accordance with any one of the embodiments, the ultrasonic energy is applied at a frequency range of about 10,000 Hz to about 100,000 Hz.

In accordance with any one of the embodiments, the frequency of the ultrasonic energy is between about 40,000 Hz to about 65,000 Hz. In some embodiments, the frequency of the ultrasonic energy is about 45,000 Hz. In some embodiments, the frequency of the ultrasonic energy is about 60,000 Hz. In some embodiments, the frequency of the ultrasonic energy is about 40,000 Hz.

In accordance with any one of the embodiments, the time or duration of the ultrasonic energy is applied is between about 5 seconds and about 120 seconds. In some embodiments, time or duration of the ultrasonic energy is applied is between 35 seconds to 60 seconds. In some embodiments, the time or duration of the ultrasonic energy is applied about 45 seconds. In some embodiments, the time or duration of the ultrasonic energy is applied is not more than 80 seconds.

In accordance with any one of the embodiments, the ultrasonic energy is concentrated from between 0.01-2.00 mm from the tip of the sonotrode of the device.

In accordance with any one of the embodiments, the subject has glaucoma or is a glaucoma suspect

In accordance with any one of the embodiments, the subject has optic neuropathies.

In accordance with any one of the embodiments, the ultrasonic energy stimulates cell growth in the RNFL by an average of at least about 1%-1000% compared to a baseline thickness of the RNFL. In some embodiments, the ultrasonic energy stimulates cell growth in the RNFL by an average of at least about 5%-100% compared to a baseline thickness of the RNFL. In some embodiments, the ultrasonic energy stimulates cell growth in the RNFL by an average of at least about 1%, 2%, 5%, 8%, 10%, 20%, 30%, 40%, 50%, 80%, 100%, 150%, 200%, 300%, 500%, 800%, or 1000%.

In accordance with any one of the embodiments, the ultrasonic energy reduces the cup to disc ratio by an average of at least about 1%-1000% compared to a baseline cup to disc ratio. In some embodiments, the ultrasonic energy reduces the cup to disc ratio by an average of at least about 5%-100% compared to a baseline cup to disc ratio. In some embodiments, the ultrasonic energy reduces the cup to disc ratio by an average of at least about 1%, 2%, 5%, 8%, 10%, 20%, 30%, 40%, 50%, 80%, 90%. In some cases, the thickness of the RNFL may be measured using optical coherence tomography.

In accordance with any one of the embodiments, the ultrasonic energy stabilizes the Vision field or increases the Vision field (VFI) by an average of at least about 1%-100% compared to a baseline VFI. In some cases, the VFI may be measured with static perimetry. In some embodiments, the ultrasonic energy increases the Vision field (VFI) by an average of at least about 10%-35% compared to a baseline VFI. In some embodiments, the ultrasonic energy increases the Vision field (VFI) by an average of at least about 10%, 20%, or 30%.

In one aspect, described herein are devices for delivering ultrasonic energy to a subject for improving vision or increasing thickness of the RNFL in accordance with any one of the embodiments.

In some embodiments, the disclosure describes a device for delivering ultrasonic energy to a subject for improving vision by the methods of the disclosure, described elsewhere herein. In some embodiments, the subject treated with the device is suffering from a eye disorder, as described elsewhere herein. In some embodiments, the device delivers ultrasonic energy to one or more ocular tissues as described elsewhere herein. In some embodiments, the device increases thickness of a retinal nerve fiber layer tissue of a subject by the methods, described elsewhere herein. In some embodiments, the device is described throughout the instant application specification and shown throughout FIGS. 8A-8C.

### 6. DETAILED DESCRIPTION

In a human clinical study described herein, we discovered that low intensity and low frequency ultrasound treatment provided stabilization of the Cup/Disc ratio, decreased Cup/Disc ratio, improved vision field, increased RNFL thickness, and/or reduced or decreased RNFL thinning in comparison to the baseline. The study suggests that the ultrasound treatment can provide neuroprotection or neuroregeneration and can be used as therapy for eye disorders, particularly those associated with retina nerve fiber layer (RNFL) thinning.

Without being bound by a theory, the mechanism of action of the ultrasound treatment can involve (1) vibrational micro-stretching of the trabecular meshwork that stimulates secretion of matrix metalloproteinase 3 (MMP-3), clearing debris and decreasing IOP, and (2) a mild, localized hyperthermic effect that induces an inflammatory response, leading to secretion of beneficial cytokines as well as clearance of debris from the trabecular meshwork by macrophages. Our ex vivo studies demonstrated increased expression of TNF-α in the trabecular meshwork in porcine eyes, and mTOR in the ciliary body and aqueous humor in bovine eyes.

Accordingly, described herein are devices, systems and methods of using the same for reducing progression of an ocular disease and/or eye disorder using ultrasound. The methods and devices find utility in the treatment of glaucoma or optic neuropathies. The methods and devices locally provide energy to the eye tissues via ultrasound. The methods and devices include the application of ultrasound from an external energy source to the outside eye tissue. The methods and devices serve to perform any one or a combination of increasing blood flow, increasing the retinal nerve fiber layer (RNFL) thickness, reducing cup to disc ratio (C/D), lowering intraocular pressure, increasing the vision field, and improving and/or preventing deterioration of ocular tissue health following single or repeated use of the apparatus. In some embodiments, the methods and devices increase RNFL thickness. In some embodiments, the method and device prevent or reduce RNFL thinning. In some embodiments, reducing progression of the ocular disease promotes or stimulates rejuvenation or growth of tissue in and around the retinal area including the optic nerve, the optic nerve head, and/or surrounding structures. In certain embodiments, the methods and device reduce progression of glaucomatous optic neuropathy.

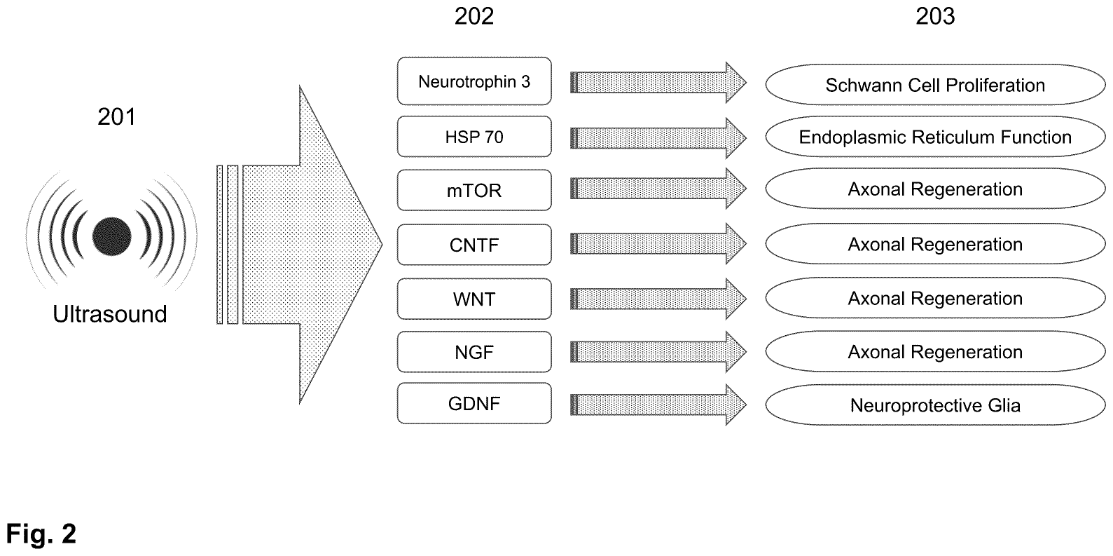

The method comprises providing an ultrasonic device that emits low powered ultrasonic energy, holding the ultrasonic device at a desired location, and transmitting the ultrasonic energy at a frequency to a desired location for a predetermined time (FIG. 1). The ultrasound energy can trigger a number of positive biochemical changes consistent with nerve regeneration and/or neuroprotection (FIG. 2), and may be used to treat specific retinal diseases such as macular degenerative, developmental retinopathies, corneal neurotrophic ulcers, and keratitis sicca. In some instances, the ultrasonic energy 105 is intended to project throughout the interior of the eye, more specifically to the retina 112, e.g., a band of the ganglion cell layer at or near the fovea and/or the cell bodies of the axons forming the optic nerve, as shown in FIG. 1. In some cases, the ultrasonic energy 105 may comprise a dispersive ultrasonic energy incident on the retina 112 and/or the choroid 114.

### 7. DEVICES AND METHODS OF USE

The device for use of any of the methods or systems described herein includes a metallic tip and ultrasound that is aimed at increasing/triggering integrins and elevating the temperature within the treatment area to a level that begins a biochemical cytokine cascade that is then absorbed systemically leading to a decrease in intraocular pressure in both eyes. In some cases, the metallic tip may be comprised of a material of titanium, stainless steel, aluminum, or any combination thereof materials. In some cases, the temperature level induced by the device and/or systems, described elsewhere herein, may, through a diffusive process transmit or transfer from an external surface of the eye to the retina. In some cases, the temperature level may (e.g., a heating effect in raising temperature) may cause a change, e.g., an upregulation or a downregulation, of one or more biochemical pathways, as described elsewhere herein, of ocular tissues e.g., retina or the retina nerve fiber layer. In some cases, the changes of the one or more biochemical pathways may be measured by detecting an expression of one or more biomarkers associated with the biochemical pathways, as described elsewhere herein. In some cases, the changes of the one or more biochemical pathways may comprise local and/or systemic changes in the one or more biochemical pathways.

In some cases, the device and/or systems for use of any of the methods described herein, may provide an acoustic radiative force (ARF) to one or more ocular tissues. In some instances, the ARF may comprise a mechanical force e.g., a pushing force, provided by one or more ultrasonic pressure waves generated by the devices and/or systems described elsewhere herein. In some cases, the ARF may change and/or induce a change to one or more biochemical pathways of one or more ocular tissues, as described elsewhere herein. In some cases, the changes to the one or more biochemical pathways may be measured by an expression of one or more biomarkers, described elsewhere herein. In some embodiments, the device is a low power, low frequency ultrasonic device. Exemplary devices and methods of use are described in U.S. Pat. Nos. 7,909,781; 8,043,235; 9,125,722, each of which is incorporated herein by reference in its entirety.

As an illustration, the device for the treatment of an eye disorder by ultrasound described below includes a balance such that the frequency, power, duty cycle, and duration of the propagated ultrasound has the optimum balance of controlled cavitation, heat and acoustic streaming to affect the trabecular meshwork. The effect is such that debris, or other occlusive structures, may be dislodged to create a larger outflow by the forces mentioned above. In some cases, the frequency, power, duty cycle, and/or the duration of the one or more propagated ultrasound waves may enable the propagation of the one or more ultrasound waves, provided by a device or system described elsewhere herein, from an external surface and/or tissue of the eye towards and/or incident on one or more internal eye tissues (e.g., the retina of the eye) thereby increasing the delivery of the ultrasound energy of the one or more ultrasound waves to the one or more internal eye tissues. In addition, the nature of the heat generated and the subsequent inflammatory reaction and integrin absorption of ultrasound with the release of cytokines directed to initiating cascades of biochemical reactions that lead to remodeling of the extracellular matrix and induction of macrophages to remove extracellular debris to further enhance the long-term effect of the treatment. It will be understood that performance of the method described herein causes an inflammatory response that causes the cells to release cytokines. The cytokines trigger enzymes and macrophage activity. The enzymes break down the extracellular debris clogging the trabecular meshwork and the macrophages clear the broken-down debris.

Described herein are two types of instruments used for ultrasonically treating the eye, one for immediately after cataract surgery (intraocular), and one for use on the surface of the eye (external), which can be used without having to enter the interior of the eye.

The device 300, as shown in FIGS. 8A-8C, for treatment on the outside surface of the eye may comprise a power cord 302, a power supply and/or an ultrasonic transducer 322 housed within a casing (306, 332). In some cases, one or more components, one or more subcomponents and/or segments of the device may be fastened using one or more fasteners (310, 330) (e.g., machine screws such as cap screws). In some cases, the power cord 302 may provide electrical coupling with a base system comprising a power supply and/or one or more processors configured to provide electrical signals to the device 300. In some cases, the power cord 302 may comprise at least 4 conductors electrically coupled to one or more electrodes 320 of the ultrasonic transducer 322 configured to deliver electrical signals to the ultrasonic transducer 322. In some cases, the electric signals delivered to the ultrasonic transducer may actuate or move the ultrasonic transducer, as described elsewhere herein. In some cases, the power cord 302 may be mechanically coupled to a strain relief boot 304 to allow for deflection and/or bending of the power cord 302 without compromising and/or attenuating an electrical signal delivered to the device when the device 300 is handled by a user during treatment the device 300. It is contemplated that either AC or DC power can be used. However, in a preferred embodiment, DC power is provided (which may be from alternating current and then converted to DC or it may be from a battery pack). It will be understood by those skilled in the art that the type of ultrasonic transducer 322 is not a limitation on the present invention. For example, the ultrasonic energy can be provided by piezoelectric materials, liquids, crystals, etc. See, for example, U.S. Pat. No. 6,616,030, which is incorporated herein by reference in its entirety. As an illustration, the ultrasonic transducer uses piezoelectric technology. The ultrasonic energy produced by the transducer may be transmitted from an ultrasound transducer 322 (e.g., a piezo electric element) to a base of the tip 325 (i.e., the sonotrode) of the device. In some instances, the tip 326 of the device contacts a surface of one or more external ocular tissues, e.g., conjunctiva and/or scleral tissue. In some cases, the ultrasound transducer 322 may be mechanically coupled to one or more rigid bodies 318. The one or more rigid bodies 318 may be made of metal (e.g., brass). The one or more rigid bodies 318 may provide a framing and/or structure allowing for the transduction and/or transfer of the motion of the actuated ultrasound transducer 322 to the tip 326 of the device. In some cases, the ultrasound transducer 322 may be mechanically coupled to the one or more rigid bodies 318 through a mounting body 324. In some cases, a rod 314 and fastening member 312 (e.g., a nut), may be mechanically coupled to the ultrasonic transducer 322. In some cases, tightening the fastening member 312 coupled to the rod 314 may pre-condition and/or pre-stress the ultrasound transducer 322 when at rest to calibrate the range of motion and/or actuation of the ultrasonic transducer 322, described elsewhere herein. In some instances, the device 300 may comprise one or more O-rings (308, 328), configured to seal one or more regions and/or segments of the device from condensation and/or liquid in contact with one or more regions and/or segments of the device. The sealing of the one or more regions and/or segments of the device may protect and maintain operability of the one or more electrical components of the device, described elsewhere herein. In some cases, the tip 326 may comprise a taper geometry where the tapered geometry may provide characteristic operating parameters of the delivery of ultrasonic energy to one or more tissues of an eye. In some cases, the tip 326 may be smooth and rounded with a surface that allows for appropriate gel or liquid interface to the ocular surface. The smooth tip 326 is preferred over the sharp tip of the prior art to prevent laceration of the exterior ocular surface or the cornea. The tip 326 may be round, circular, and/or flat. In some cases, the tip 326 may comprise up to about a 3 mm diameter. In some cases, the tip 326 of the device 300 may comprise a radius of at least about 1 mm at the cut edge of tip. In some cases, the curvature and/or geometry of the tip may provide coupling of at least about 75%, 80%, 85%, 90%, 95% of the one or more ultrasonic waves generated by the ultrasound transducer to one or more ocular tissues. In some cases, the length of the tip (334, 336) may amplify actuated movement of the ultrasound transducer 322. In some cases, the tip 326 may comprise a length as long as half the wavelength of ultrasonic energy outputted by the tip.

In an exemplary embodiment, the casing is attached to the transducer at a null point to not upset or diminish ultrasound production within the casing; but avoiding contact with the tip to allow maximum energy. There may be a space between the casing and rod and/or tip. The casing can be attached to the transducer, for example, by threaded fasteners, rivets or the like.

The casing may be shaped so that it fits easily into a user's hand. In a preferred embodiment, the casing includes a handle extending therefrom that can be grasped by a user's second hand. With this design the user can grasp the casing with one hand and use the other hand to guide the device using the handle. This provides a greater ability to manipulate the device as desired. The handle may be straight or bent. The casing may also include a depression or depressions therein or other ergonomic additions to make the casing easier to grip.

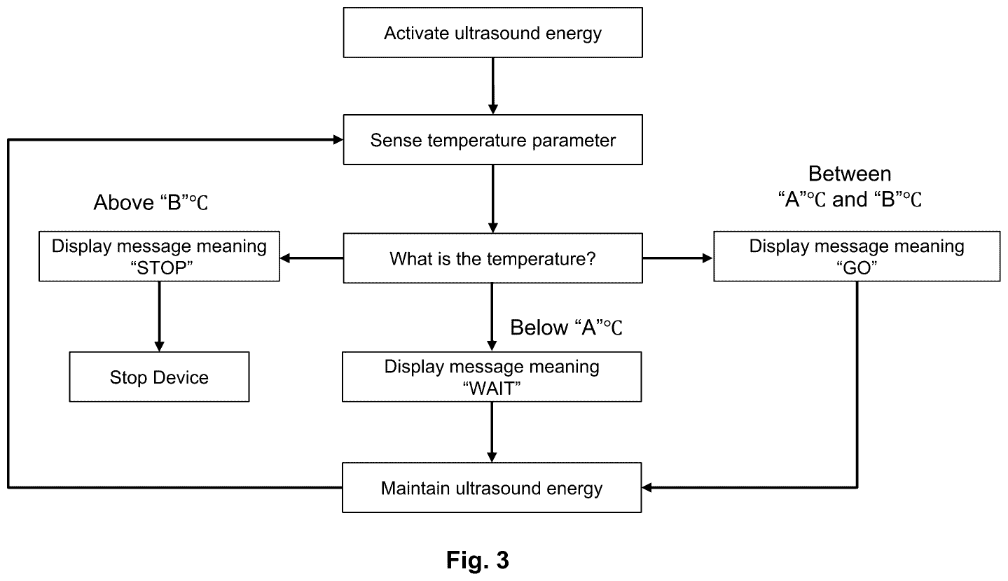

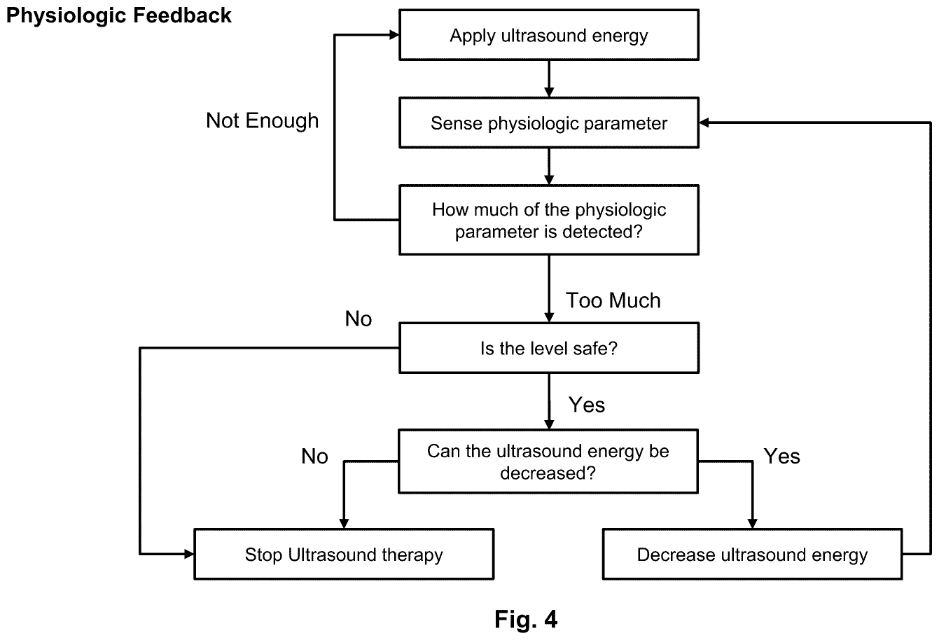

Several features of the therapeutic device include: a topically applied low power ultrasound, a feedback system, and a monitoring system to display such feedback. The feedback system may be used to verify application specific, device status (FIG. 3), and physiologic parameters (FIG. 4). Primarily, the feedback is acquired via sensors. The sensors may be attached to the device, at a location external to the device but near the point of contact of the therapy, and locations at a distance from the application of therapy.

Feedback may be acquired from imaging devices and systems integrated and/or not attached to the device. In some cases, the feedback may be measured by e.g., a camera (e.g., a thermal infrared camera), an optical coherence tomography (OCT) probe and/or other modalities that detect and/or determine whether or not ultrasonic energy is provided to a target one or more ocular tissues, for example, toward the retina, ciliary body, or trabecular meshwork. For example, a thermal infrared camera may detect and/or measure one or more temperatures of one or more ocular tissues as ultrasonic energy is directed towards and/or is incident on the one or more ocular tissues. In some instances, the thermal infrared camera may measure such signals in real-time e.g., at or greater than 30 frames per second. In some instances, the real-time imaging speed may provide feedback to a user of the device to adjust a position of the device during and/or after treatment.

In some cases, an OCT probe may provide data of one or more images of one or more tissues of the eye, where the one or more images provide a position feedback of the device with respect to one or more landmarks or ocular tissues. In some instances, the position feedback may be used to adjust a suggested position (e.g., a three-dimensional cartesian coordinate of a reference point) of the device to provide and/or transmit ultrasonic energy of the device to one or more target ocular tissues. In some cases, an OCT probe may provide data of one or more images of one or more ocular tissues, where the data of the one or more images indicate a change in anatomical structure of the one or more ocular tissues indicative of ultrasonic energy absorbed by the one or more ocular tissues. In some cases, the one or more images indicating absorption of the ultrasonic energy may be used in within a feedback loop in adjusting one or more parameters of the device, as described elsewhere herein. In some cases, the adjustment of the one or more parameters may be conducted in real time. In conjunction with the imaging device may be a fixation target for the patient to help the user with positioning of the device and its aiming characteristics.

In some cases, the feedback system may also adjust functional settings of the device without user input; for example, the device may calibrate automatically to predetermined parameters.

Application specific feedback and monitoring may include the sensing of location and depth of the ultrasound energy and force feedback to verify proper application of the device to the target tissue. Device status feedback and monitoring may include temperature sensing of the transducer and energy delivery from the transducer such as by measuring the reflected ultrasound from the target tissue. Physiologic parameter feedback may include remote or direct contact temperature sensing, movement, visual.

The monitoring system may include for example, a display, blinking lights of different colors similar to a tuning instrument, audible cues, mechanical/vibratory cues, and so forth. The monitoring system may be included as part of the handpiece or located on the console or both.

In operation, the ultrasonic energy is provided with an input power at a frequency for a period of time. As is described herein, these ranges can be different for individual cases.

The frequency range of the ultrasonic energy provided by the devices and/or systems, described elsewhere herein, may be about 10,000 to 500,000 Hz. In some embodiments, the frequency range may be from about 30,000 to 100,000 Hz. In some embodiments, the frequency range may be from about 30,000 to 50,000 Hz. In some embodiments, the frequency range may be from about 35,000 to 45,000 Hz. In some embodiments, the frequency of the ultrasonic energy may be about 10,000, 20,000, 25,000, 30,000, 35,000, 40,000, 45,000, 50,000, 55,000, 60,000, 65,000, 70,000, 75,000, 80,000, 85,000, 90,000, 95,000, or 100,000 Hz. In some embodiments, the frequency of the ultrasonic energy may be about 40,000 Hz. In some embodiments, the frequency of the ultrasonic energy may be about 45,000 Hz. In some embodiments, the frequency of the ultrasonic energy may be about 60,000 Hz.

The duration of applying ultrasonic energy with the device and/or systems, described elsewhere herein, may comprise about 5 to about 120 seconds. In some embodiments, the duration of applying ultrasonic energy may comprise about 25 to about 60 seconds. In typical embodiments, the duration of applying ultrasonic energy may comprise about 40 to about 50 seconds. In some embodiments, the ultrasonic energy is provided for about 5, 10, 15, 20, 25, 30, 35, 40, 45, 50, 55, 60, 65, 70, 75, 80, 85, 90, 95, 100, or 120 seconds. In some embodiments, the ultrasonic energy is provided for about 30 seconds. In some embodiments, the ultrasonic energy is provided for about 45 seconds. In some embodiments, the ultrasonic energy is provided for about 60 seconds. In some cases, providing the ultrasonic energy for at least about 40 seconds may result in a change of one or more biochemical pathways and/or production or expression of one or more biomarkers, as described elsewhere herein. In some instances, providing the ultrasonic energy for up to about 50 seconds may provide a window for ultrasonic energy to be delivered and/or provided to one or more ocular tissues without causing cellular death of the one or more ocular tissues.

The input power of the devices and/or systems, described elsewhere herein, may be in the range of about 0.1 to about 60 watts. In some embodiments, the input power may be provided in the range of about 1 to about 6 watts. In some certain embodiments, the input power may be provided in the range of about 3 to about 4 watts.

In some embodiments, the input power may be sufficient to generate about 1 to about 30 watts of acoustic power. In some embodiments, the input power may be sufficient to generate about 1 to about 6 watts of acoustic power. In some embodiments, the input power may be sufficient to generate about 0.1 to about 3 watts of acoustic power. In some embodiments, the input power may be sufficient to generate about 0.5 to about 2 watts of acoustic power. In some embodiments, the input power may be sufficient to generate about 1 to about 3 watts of acoustic power. In some embodiments, the input power may be sufficient to generate about 1.5 to about 2.0 watts of acoustic power. In some embodiments, the input power may be sufficient to generate about 0.5 to about 3.99 watts of acoustic power. In some embodiments, the input power may be sufficient to generate about 2.0 to about 3.99 watts of acoustic power. In some embodiments, the input power may be sufficient to generate about 3.5 watts of acoustic power. In some embodiments, the input power may be sufficient to generate about 3 watts of acoustic power.

In some embodiments, the effective ultrasonic intensity from the ultrasonic device has about 0.1 watts/cm², about 0.2 watts/cm², about 0.3 watts/cm², about 0.4 watts/cm², about 0.5 watts/cm², about 0.6 watts/cm², about 0.7 watts/cm², about 0.8 watts/cm², about 0.9 watts/cm², about 1.0 watts/cm², about 1.1 watts/cm², about 1.2 watts/cm², about 1.3 watts/cm², about 1.4 watts/cm², about 1.5 watts/cm², about 1.6 watts/cm², about 1.7 watts/cm², about 1.8 watts/cm², about 1.9 watts/cm², about 2 watts/cm², about 2.2 watts/cm², about 2.5 watts/cm², about 2.8 watts/cm², or about 3.0 watts/cm².

In some embodiments, the ultrasonic energy concentrated at the distance from the tip of the sonotrode of the ultrasonic device is about 4 watts. In some embodiments, the ultrasonic energy concentrated at the distance from the tip of the sonotrode of the ultrasonic device is about 2 watts. In some embodiments, the ultrasonic energy concentrated at the distance from the tip of the sonotrode of the ultrasonic device is less than 2 watts. In some embodiments, the ultrasonic energy concentrated at the distance from the tip of the sonotrode of the ultrasonic device is about 1.8 watt.

In some embodiments, the ultrasonic intensity is concentrated at a distance between 0.01-2.00 mm, 0.01-10 mm, 0.5-5 mm, or 1-3 mm from the tip of the sonotrode of the ultrasonic device. In some embodiments, the ultrasonic intensity is concentrated at a distance about 0.5 mm, about 1 mm, about 1.5 mm, or about 2 mm from the tip of the sonotrode of the device.

In some embodiments the ultrasonic energy is applied as 10 to 15 applications. In some embodiments the ultrasonic energy is applied as 10, 11, 12, 13, 14, or 15 applications. In some embodiments, each of the applications lasts between 5 seconds and 120 seconds.

In some embodiments, the ultrasound is applied while moving the tip of the sonotrode in one direction on the limbus. In some embodiments, the ultrasound is applied while moving the tip of the sonotrode in the clockwise or counterclockwise direction. In some embodiments, the ultrasonic energy is applied as 12 applications at 12 clock hour positions on the limbus.

In some embodiments, the ultrasound energy is provided at 45 degrees with respect to a normal vector of a surface of a limbus. In some embodiments, the ultrasound energy is provided at 40 to 60 degrees with respect to a normal vector of a surface of a limbus. In some embodiments, the ultrasound energy is provided at 40 to 50 degrees with respect to a normal vector of a surface of a limbus. In some embodiments, the ultrasound energy is provided at 50 to 60 degrees with respect to a normal vector of a surface of a limbus.

In some embodiments, the ultrasonic energy is provided toward the trabecular meshwork of the subject's eye.

The ultrasonic device may also include a power source that is rechargeable and can be recharged with an external charging station, or that is disposable and consists of replaceable lithium-ion or other sources of direct current (i.e., batteries). The device may also be powered by alternating current from an external source (e.g., an electrical outlet).

The rechargeable ultrasonic device may hold a charge allowing for one or more treatments before requiring recharging. The ultrasonic device may also include feedback for alerting the user as to when the treatment duration is finished and/or when there is too high a temperature at the ultrasound transducer surface (for safety) including but not limited to visual indicators (light emitting diodes or electric lamps), vibratory motors which pulse for a short duration of time or auditory or vibratory signals. Additional signals may also be provided to notify the user that the handheld ultrasonic device requires recharging.

The methods associated with the handheld ultrasonic devices may achieve one or more of the following effects on ocular tissue: increase cellular calcium uptake, increase cellular activity, increase cell metabolism, increase protein synthesis by fibroblasts, promote collagen synthesis and deposition, promote cell proliferation, promote cell degranulation, increase synthesis of non-collagenous protein (NCP), increase production and signaling of Vascular Endothelial Growth Factor (VEGF), stimulate the formation of endothelial cells, stimulate the release of endothelial growth factors, promote angiogenesis, increase in angiogenesis-related chemokines or cytokines (e.g., mTOR, Interleukin 8, IL-8, IL-1, IL-6, or basic Fibroblast Growth Factor, bFGF, or TNF-alpha), and/or reduce neurodegenerative biochemicals (e.g., BDNF, NGF, nt3, GDNF, ARTEMIN, PERSEPHIN, Neurotactin, Granulocyte-Macrophage Colony Stimulating Factor (GM-CSF), endothelial NOS (enos), neuronal NOS (nNOS), CNTF, and/or neurokinin). In some cases, CNTF may be used to treat dry age-related macular degeneration (AMD). In some embodiments, the effects on ocular tissue are observed in the treated eye and/or the contralateral eye. See U.S. Pat. No. 8,043,235, which is incorporated herein by reference in its entirety. In some embodiments, the devices and methods of use of the devices for treating an ocular disorder increases TNF-alpha in the trabecular meshwork.

The ultrasound energy source may be a piezoelectric (PZT) ceramic or electromagnetic transducer. A wave generator may be disposed within the device or in a separate apparatus. The PZT may be constructed from piezoelectric materials such as lead zirconate titanate, potassium niobate, sodium tungstate, etc. The transducer assembly may consist of either one or an array of piezo-ceramic ultrasound transducers. The transducer assembly may also be Capacitive Micromachined Ultrasound Transducers (CMUT) to appropriately apply diffuse, focused, or unfocused ultrasound or provide constructive ultrasound wave interference and direct the ultrasound energy to the target tissue. The target of the ultrasound energy (unfocused or focused) may be further tuned to cover tissue of the retina 112 and underlying tissues; for example, the choroid 114. In some cases, the transducer may be tuned to provide a point source energy of up to about 1 mm from the tip of the device. In some cases, tuning the transducer may comprise adjusting and/or changing a frequency of the ultrasonic wave provided and/or emitted by the transducer. In some cases, the tuning of the device may be determined such that the point source of energy of the device may be within the angled structure of the anterior chamber. In some instances, the ultrasound transducer may have an effective radiating area between 0.1 cm² and 10 cm². Although the ultrasonic energy is provided to superficial tissues of the eye, e.g., the conjunctiva, sclera, and trabecular meshwork, it is unexpected that the unfocused ultrasonic energy traverses through the globe of the eye to the retina tissues, inducing an effect of neuro-preservation and/or regeneration of tissue of the retina nerve fiber layer of the retina.

The ultrasonic device may have a duty cycle of anywhere between 20% and 100%. The term duty cycle refers to the percentage of time that a pulsed ultrasound wave is on (e.g., a 50% duty cycle means that a pulsed wave is on 50% of the time). At a duty cycle of 100% (also called a continuous duty cycle), the pulsed wave is on 100% of the time. The intensity and duty cycle can either be individually set for each treatment or set once for all subsequent treatments. In some cases, the intensity and duty cycle may be set for a subject based at least on a thickness of scleral tissue of the subject. In some cases, the thickness of scleral tissue may be measured with OCT. The intensity and duty cycle may be automatically set as a feature pre-programmed into the device and may or may not change. In some embodiments, the intensity and duty cycle settings are changed based on previous treatment duration and results.

### 8. TREATMENT

In one aspect, provided are methods for treating a subject suffering from or at risk of an eye disorder, the method comprising administering providing ultrasonic energy to the limbus of an eye at an effective treatment schedule using an ultrasonic device that emits ultrasonic energy of between 0.01-5 watts of power.

In one aspect, provided are methods for increasing thickness of RNFL in a subject, the method comprising administering providing ultrasonic energy to the limbus of an eye at an effective treatment schedule using an ultrasonic device that emits ultrasonic energy of between 0.01-5 watts of power.

### 8.1.1 Treatment Schedule

In various embodiments, ultrasound treatment is applied to the patient in need thereof in one or more session to deliver ultrasound energy to the patient's eye.

In some embodiments, the effective treatment schedule comprises two or more sessions of providing ultrasonic energy to the limbus of an eye. The ultrasonic energy can be provided toward the trabecular meshwork of the subject's eye. In some embodiments, the effective treatment schedule comprises two sessions of providing ultrasonic energy to the limbus of an eye. In some embodiments, the two sessions are scheduled 1 month, 3 months, 6 months, 12 months, 18 months, 24 months, 36 months, or 48 months apart from each other. In some embodiments, the effective treatment schedule comprises three sessions of providing ultrasonic energy to the limbus of an eye.

In some embodiments, the effective treatment schedule provides ultrasonic energy to the limbus of the eye every 1 month, 3 months, 6 months, 12 months, 18 months, 24 months, 36 months, or 48 months, or longer.

In some embodiments, the effective treatment schedule provides ultrasonic energy to the limbus of the eye over a period of 1 month, 3 months, 6 months, 12 months, 18 months, 24 months, 36 months, or 48 months, or longer.

### 8.1.1.1 Stimulation of Cell Growth in the RNFL

In some embodiments, the effective treatment schedule is sufficient to modulate one or more biomarkers selected from mammalian target of rapamycin (mTOR), wingless-related integration site (WNT), nerve growth factor (NGF), glial cell line-derived neurotrophic factor (GDNF), brain-derived neurotrophic factor (BDNF), interleukin 1 (IL-1), interleukin 6 (IL-6), interleukin 8 (IL-8), tumor necrosis factor alpha (TNF-α), matrix metalloproteinase 3 (MMP-3), Ak strain transforming 1 (Akt1), paired box 6 (Pax6), B-cell leukemia/lymphoma 2 (Bcl-2), ciliary neurotrophic factor (CNTF), heat shock protein 70 (HSP70), and brain-specific homeobox/POU domain protein 3 (Brn3a).

In some embodiments, the one or more biomarkers are selected from brain-derived neurotrophic factor (BDNF), nerve growth factor (NGF), glial cell line-derived neurotrophic factor (GDNF), matrix metalloproteinase 3 (MMP-3), mammalian target of rapamycin (mTOR), tumor necrosis factor alpha (TNF-α), and combinations thereof. In some embodiments, the biomarkers are increased in the subject's aqueous humor, trabecular meshwork, ciliary body, retina, or combinations thereof, by at least 10%, at least 20%, at least 30%, at least 40%, at least 50%, at least 60%, at least 70%, at least 80%, at least 90%, or 100%, at least 200%, at least 300%, at least 400%, at least 500%, at least 1000%, or more as compared to baseline. In some embodiments, the effective treatment schedule increases TNF-α in the trabecular meshwork. In some embodiments, the effective treatment schedule increases mTOR in the aqueous humor. In some embodiments, the effective treatment schedule increases mTOR in the ciliary body.

In some embodiments, the biomarkers are decreased in the subject's aqueous humor, trabecular meshwork, ciliary body, retina, or combinations thereof, by at least 10%, at least 20%, at least 30%, at least 40%, at least 50%, at least 60%, at least 70%, at least 80%, at least 90%, or 100%, at least 200%, at least 300%, at least 400%, at least 500%, at least 1000%, or more as compared to baseline. In some embodiments, the effective treatment schedule decreases mTOR in the retina. In some embodiments, the effective treatment schedule decreases mTOR in the trabecular meshwork.

In some embodiments, the effective treatment schedule is sufficient to increases expression level of biomarkers selected from neurotrophin 3, CNTF, HSP70, or any combination thereof by at least 10%, at least 10%, at least 30%, at least 40%, at least 50%, at least 60%, at least 70%, at least 80%, at least 90%, or 100%, at least 200%, at least 300%, at least 400%, at least 500%, at least 1000%, or more as compared to baseline.

In some embodiments, the effective treatment schedule is sufficient to decrease expression level of biomarkers selected from neurotrophin 3, CNTF, HSP70, or any combination thereof by at least 10%, at least 10%, at least 30%, at least 40%, at least 50%, at least 60%, at least 70%, at least 80%, at least 90%, or 100%, at least 200%, at least 300%, at least 400%, at least 500%, at least 1000%, or more as compared to baseline.

In some embodiments, the effective treatment schedule is sufficient to increase gene expression level of mTOR, WNT, NGF, GDNF, BDNF, IL-1, IL-6, IL-8, TNF-alpha, Akt1, Pax6, Bcl-2, and Brn3a in retinal ganglion cells (RGC) by at least 10%, at least 10%, at least 30%, at least 40%, at least 50%, at least 60%, at least 70%, at least 80%, at least 90%, or 100%, at least 200%, at least 300%, at least 400%, at least 500%, at least 1000%, or more as compared to baseline.

In some embodiments, the effective treatment schedule is sufficient to decrease gene expression level of mTOR, WNT, NGF, GDNF, BDNF, IL-1, IL-6, IL-8, TNF-alpha, Akt1, Pax6, Bcl-2, and Brn3a in retinal ganglion cells (RGC) by at least 10%, at least 10%, at least 30%, at least 40%, at least 50%, at least 60%, at least 70%, at least 80%, at least 90%, or 100%, at least 200%, at least 300%, at least 400%, at least 500%, at least 1000%, or more as compared to baseline.

In some embodiments, the effective treatment schedule is sufficient to increase protein expression level of mTOR, WNT, NGF, GDNF, BDNF, IL-1, IL-6, IL-8, TNF-alpha, Akt1, Pax6, Bcl-2, and Brn3a in retinal ganglion cells (RGC) by at least 10%, at least 10%, at least 30%, at least 40%, at least 50%, at least 60%, at least 70%, at least 80%, at least 90%, or 100%, at least 200%, at least 300%, at least 400%, at least 500%, at least 1000%, or more as compared to baseline.

In some embodiments, the effective treatment schedule is sufficient to decrease protein expression level of mTOR, WNT, NGF, GDNF, BDNF, IL-1, IL-6, IL-8, TNF-alpha, Akt1, Pax6, Bcl-2, and Brn3a retinal ganglion cells (RGC) by at least 10%, at least 10%, at least 30%, at least 40%, at least 50%, at least 60%, at least 70%, at least 80%, at least 90%, or 100%, at least 200%, at least 300%, at least 400%, at least 500%, at least 1000%, or more as compared to baseline.

In some embodiments, the effective treatment schedule is sufficient to increase axonal growth in the RNFL or thickness of the RNFL by at least 10%, at least 10%, at least 30%, at least 40%, at least 50%, at least 60%, at least 70%, at least 80%, at least 90%, or 100%, at least 200%, at least 300%, at least 400%, at least 500%, at least 1000%, or more as compared to baseline.

### 8.1.1.2 Increase of RNFL Thickness or Prevention of RNFL Thinning

In some embodiments, the effective treatment schedule is sufficient to increase axonal growth in the RNFL or thickness of the RNFL. The RNFL thickness can measured by Heidelberg Retinal Tomography (HRT) or optical coherence tomography (OCT).

In some embodiments, ultrasound treatment increases RNFL thickness by at least at least 10%, at least 10%, at least 30%, at least 40%, at least 50%, at least 60%, at least 70%, at least 80%, at least 90%, or 100%, at least 200%, at least 300%, at least 400%, at least 500%, at least 1000%, or more as compared to baseline in treatment naïve patients, patients whose eye disorder progressed after treatment, or patients who are on one or more medications.

In some embodiments, ultrasound treatment provides neuroprotection and prevents RNFL thinning by at least 10%, at least 10%, at least 30%, at least 40%, at least 50%, at least 60%, at least 70%, at least 80%, at least 90%, or 100%, at least 200%, at least 300%, at least 400%, at least 500%, at least 1000%, or more as compared to baseline in treatment naïve patients, patients whose eye disorder progressed after treatment, or patients who are on one or more medications.

In some embodiments, ultrasound treatment increases RNFL thickness and/or prevents RNFL thinning in at least 30%, at least 40%, at least 50%, at least 60%, at least 70%, at least 80%, at least 90%, or 100% of patients.

In some embodiments, ultrasound treatment increases RNFL thickness and/or prevents RNFL thinning in the patients' eye for at least 1 month, 2 months, 3 months, 4 months, 5 months, 6 months, 7 months, 8 months, 9 months, 10 months, 11 months, 12 months, 18 months, 24 months, or longer.

In some embodiments, the effective treatment schedule is sufficient to increase the RNFL thickness in both treated and untreated eyes. The ultrasonic energy can be applied to one or both eyes.

In some embodiments, the effective treatment schedule is sufficient to decrease a cup to disc ratio of the eye by at least 5%, at least 10%, at least 20%, at least 30%, at least 40%, at least 50%, at least 60%, at least 70%, at least 80%, at least 90%, or 100% as compared to baseline. The ratio can be measured by Optical coherence tomography (OCT) or Heidelberg Retinal Tomography (HRT).

### 8.1.1.3 Reduction of IOP

In some embodiments, the effective treatment schedule is sufficient to reduce intraocular pressure (IOP) in the subject's eye. The effective treatment schedule is sufficient to reduce the IOP by at least 20%, at least 25%, at least 30%, at least 35%, at least 40%, at least 45%, or at least 50%, at least 60%, at least 70%, at least 80%, at least 90%, or 100%, as compared to baseline. In some embodiments, the effective treatment schedule is sufficient to reduce the IOP to the level of a healthy eye or lower.

In some embodiments, ultrasound treatment as a monotherapy provides at least 5%, at least 10%, at least 20%, at least 30%, at least 40%, at least 50%, at least 60%, at least 70%, at least 80%, at least 90%, or 100% reduction in IOP as compared to baseline in treatment naïve and/or patients whose eye disorder progressed following treatment (e.g., medication).

In some embodiments, ultrasound treatment as an ancillary treatment in combination with one or more glaucoma treatments provides at least 5%, at least 10%, at least 20%, at least 30%, at least 40%, at least 50%, at least 60%, at least 70%, at least 80%, at least 90%, or 100% reduction as compared to baseline in IOP in patients who are on one or more glaucoma treatments. In some embodiments, the glaucoma treatment is a pharmaceutical agent, laser treatment, and/or surgery. In some embodiments, ultrasound treatment reduces the number of pharmaceutical agent (e.g., medication) needed to control the patient's IOP by at least at least 10%, at least 10%, at least 30%, at least 40%, at least 50%, at least 60%, at least 70%, at least 80%, at least 90%, or at least 100% as compared to baseline. In some embodiments, ultrasound treatment reduces the frequency of application of the glaucoma treatment by at least at least 10%, at least 10%, at least 30%, at least 40%, at least 50%, at least 60%, at least 70%, at least 80%, at least 90%, or at least 100% as compared to baseline.

In some embodiments, ultrasound treatment reduced IOP in at least 30%, at least 40%, at least 50%, at least 60%, at least 70%, at least 80%, at least 90%, or 100% of patients.

In some embodiments, ultrasound treatment reduced IOP in the patients' eye for at least 1 month, 2 months, 3 months, 4 months, 5 months, 6 months, 7 months, 8 months, 9 months, 10 months, 11 months, 12 months, 18 months, 24 months, or longer.

In some embodiments, ultrasound treatment maintains the patient's IOP at a level less than 21 mmHg.

8.1.1.4 Improvement of Vision Field

In some embodiments, the effective treatment schedule is sufficient to increase visual field of the eye. In some embodiments, ultrasound treatment improves a patient's vision field by at least at least 10%, at least 10%, at least 30%, at least 40%, at least 50%, at least 60%, at least 70%, at least 80%, at least 90%, or 100%, at least 200%, at least 300%, at least 400%, at least 500%, at least 1000%, or more as compared to baseline in treatment naïve patients, patients whose eye disorder progressed after treatment, or patients who are on one or more medications.

In some embodiments, ultrasound treatment recovers a patient's vision by at least at least 10%, at least 10%, at least 30%, at least 40%, at least 50%, at least 60%, at least 70%, at least 80%, at least 90%, or 100%, at least 200%, at least 300%, at least 400%, at least 500%, at least 1000%, or more as compared to baseline in treatment naïve patients, patients whose eye disorder progressed after treatment, or patients who are on one or more medications. In some embodiments, ultrasound treatment reduces paracentral vision loss and recovers paracentral vision.

In some embodiments, ultrasound treatment improves vision field, reduces paracentral vision loss, and/or recovers paracentral vision in at least 30%, at least 40%, at least 50%, at least 60%, at least 70%, at least 80%, at least 90%, or 100% of patients.

In some embodiments, ultrasound treatment vision field, reduces paracentral vision loss, and/or recovers paracentral vision in the patients' eye for at least 1 month, 2 months, 3 months, 4 months, 5 months, 6 months, 7 months, 8 months, 9 months, 10 months, 11 months, 12 months, 18 months, 24 months, or longer.

### 8.1.1.5 Subjects

Subjects who can benefit from the methods and devices described can one or more eye disorders. In some embodiments, the subject has glaucoma, has been diagnosed of having glaucoma, suffers from glaucoma symptoms, or is at risk of having glaucoma. In some embodiments, the subject has glaucoma. In some embodiments, the subject has open angle glaucoma or primary open angle glaucoma (POAG). In some embodiments, the subject has acute angle-closure glaucoma. In some embodiments, the subject has ocular hypertension.

In some embodiments, subject has not been diagnosed with glaucoma or is not suspected of having glaucoma.

In some embodiments, the subject has advanced visual disease, elevated intraocular pressure, diurnal intraocular pressure (IOP), old age, decreased central corneal thickness, disc hemorrhage, genetic mutations, large beta zone of peripapillary atrophy, or optic neuropathies.

In some embodiments, the subject has elevated intraocular pressure. The subject can have an IOP of between about 22 mmHg to about 35 mmHg before ultrasonic treatment.

In some embodiments, the subject does not have elevated intraocular pressure. In some embodiments, the subject has an IOP lower than 22 mmHg and has glaucoma symptoms. In some embodiments, the subject has normotensive glaucoma.

In some embodiments, the subject has retinal nerve fiber layer (RNFL) thinner than 80 μm, thinner than 70 μm, thinner than 60 μm, thinner than 50 μm, thinner than 40 μm or thinner than an average RNFL thickness in a healthy adult. In some embodiments, the subject has an eye disorder associated with retinal nerve fiber layer (RNFL) thinning. In some embodiments, the subject exhibits a prognosis indicating a loss of RNFL thickness.

In some embodiments, the subject has anemia, multiple sclerosis, age-related RNFL loss, retinitis pigmentosa, anterior ischemic optic neuropathy, high myopic retinal degeneration, macular degeneration, such as dry macular degeneration, wet macular degeneration, or geographic atrophy.

### 8.1.1.5.1 Other Treatments

In some embodiments, the ultrasound treatment disclosed herein is combined with other treatment method known in the art.

In various embodiments, the subject has not received alternative treatment before the ultrasonic treatment. In some embodiments, the alternative treatment is selected from intervention surgery and pharmaceutical treatments. Exemplary intervention surgery includes trabeculectomy, glaucoma implant surgery, minimally invasive glaucoma surgery (MIGS), cyclophotocoagulation, EX-PRESS mini-shunt, Ahmed Glaucoma Valve (AGV), Descemet's Membrane Endothelial Keratoplasty (DMEK), selective laser trabeculoplasty (SLT), and/or combinations thereof.

In some embodiments, the subject has not received alternative treatment before the ultrasonic treatment. Exemplary pharmaceutical agents include β-blockers, carbonic anhydrase inhibitors, prostaglandin analog, a2-adrenergic agonists, parasympathomimetic drugs, netarsudil, latanoprostene bunod, and/or combinations thereof.

In some embodiments, the subject has received one or more alternative treatments before the ultrasonic treatment. The effective treatment schedule can be sufficient to reduce need for the alternative glaucoma treatment.

### 9. EXAMPLES

The devices, methods and systems described herein are further described through reference to the following experimental examples. These examples are provided for purposes of illustration only and are not intended to be limiting.

### 9.1 Example 1. Ultrasound Treatment Reduced Progression of RNFL Thinning, and Cup to Disc Ratio

This example evaluates the potential for a low power, low frequency non-invasive ultrasound to offer neuroprotection to glaucoma patients, patients with various retinopathies, and patients with ophthalmic or eye disorder or conditions associated with elevated IOP.

### 9.1.1. Methods

Data from patients who were treated with the ultrasonic device in two clinical studies (Clinical Study I and Clinical Study II) were retrospectively reviewed.

### 9.1.1.1 Clinical Study I

In a two-branch clinical trial (Clinical Study I, registered clinical trial ISRCTN50904302), a total of 26 primary open-angle glaucoma patients underwent a procedure consisting of the external application of the ultrasonic device. In branch 1, nine of these patients were naïve to pharmaceutical treatment or had been off of medication for over 6 months. In branch 2, 17 patients were treated after a medication washout period. Patients had drug holiday for at least 4 weeks in accordance with general practice (see e.g., Diaconita et al. Washout Duration of Prostaglandin Analogues: A Systematic Review and Meta-analysis. J Ophthalmol. 2018 Sep. 27; 2018:3190684). All patients in the study were followed for at least 12 months. See also e.g., Schwartz, D. (2014). Therapeutic Ultrasound for Glaucoma (TUG). In: Samples, J. R., Ahmed, I.I.K. (eds) Surgical Innovations in Glaucoma. Springer, New York, NY. Doi.org/10,1007/978-1-4614-8348-9_12, which is hereby incorporated by reference in its entirety.

These patients were diagnosed with open angle glaucoma or were glaucoma suspects with elevated intraocular pressure (i.e., treatment naïve patients). As described herein, the term “treatment naïve patient” refers to patients who have not been treated with an alternative treatment, such as intervention surgery and/or a pharmaceutical agent at least 6 months prior to ultrasound treatment.

They had been treated with an unfocused ultrasound probe in accordance with some embodiments described herein, with a 40 KHz frequency operating frequency, less than or equal to 2 Watts of output power, and a 100% duty cycle. Unless stated otherwise, the term “ultrasound treatment” refers to a session of providing ultrasonic energy to a total of 12 spatially discreet locations around the subject's corneal limbus. Each location was treated for 45 seconds. For each discreet location treatment, the ultrasound probe was held at 45 degrees with respect to a normal vector of a surface of the limbus. Each patient may receive one or more ultrasound treatments with an interval or frequency of treatment at about every 1-6 months, or 1, 2, 3, 4, 5, 6 (e.g., 4 months), depending on the subject's IOP level at the time of visit as compared to their baseline IOP level. For example, the subject may have an IOP of about between about 22 mmHg to about 35 mmHg before ultrasonic treatment (baseline). The subject's IOP may decrease lower than 22 mmHg after an ultrasound treatment. The subject's IOP may regain to over 22 mmHg between the follow up visits (e.g., 1, 2, 3, 4, 5, 6, 7, 8, 9, 10, 11, or 12 months). In such case, the patient was given an additional ultrasound treatment until the IOP raised to an uncontrollable level (e.g., maintained at a level over 22 mmHg) at the next visit or when the patient dropped out for reasons unrelated to the efficacy of ultrasound treatment.

The ultrasound treatment utilized a sine wave form output of ultrasonic energy. The studies evaluated the effect of low power ultrasound on intraocular pressure. The protocol for these studies required the treatment of one eye and using the non-treated (contralateral) eye as a control. A frequent marker for the progression of glaucomatous loss is the thickness of the retinal nerve fiber layer (RNFL). RNFL thickness was measured using a Heidelberg retina tomograph (HRT) or an optical coherence tomography (OCT) instrument. RNFL was measured at baseline and at a follow-up visit (e.g., 1, 2, 3, 4 5 visits with an interval of about 1-24 months or 1-6 years).

### 9.1.1.2 Clinical Study II

In a separate clinical study (Clinical Study II) was designed to determine an ancillary effect of the ultrasound treatment in eyes. The study was a 6-month prospective study with a single treatment in one eye. The pharmaceutical agent was continued throughout the six months. Typically, the pharmaceutical agent is a prostaglandin analog (e.g., latanoprost) alone or added topical dorzalamide, brimonidine or alpha adrenergic.

A total of 11 patients were followed for at least 6 months. The patients were non-randomized, but part of an ongoing evaluation of the ultrasonic device. These were the first 11 patients whose intraocular pressure appeared to be inadequately controlled by a single pharmaceutical eye drop who elected to have the ultrasound treatment procedure as an ancillary treatment for better intraocular pressure control. Patients were followed for at least 6 months. The intraocular pressure control at the time of treatment ranged from 13 to 27 mmHg. Only one eye was treated under International Review Board (IRB) protocol.

The patients were evaluated at the slit lamp before the treatment and were free of infection or inflammation. Applanation intraocular pressure was measured by two persons with the results masked and an average taken after the second reading. The ultrasound treatment was done under protocol as described in the Clinical Study I.

The patient was then seen 2 hours after the treatment to check for any pressure spike. The follow-up visits were at the one day, one week and then monthly for the six months of the study. Evaluation was performed at the slit lamp for signs or symptoms of inflammation and intraocular pressure. The intraocular pressure was then taken in the same manner of two masked evaluators and an average then taken as the result.

In total, 56 patients participated in the studies. Table 1 summarizes patient profiles.

TABLE 1
Clinical study patient profile

| | | | | | Number | RNFL |
|---|---|---|---|---|---|---|
| | | | | | of | assessment |
| | Clinical Study I | | Clinical | Normo- | ultrasound | (HRT |
| Patient # | Naïve | Washout | Study II | tensive | treatment | or OCT) |
| 202 | | X | | | 3 | H |
| 216 | X | | | X | 3 | H |
| 229 | | X | | | 1 | O |
| 236 | | X | | | 1 | H |
| 237 | | X | | | 1 | H |
| 243 | | X | | X | 2 | H |
| 249 | | X | | | 1 | H |
| 251 | | X | | X | 1 | H |
| 252 | X | | | | 1 | H |
| 254 | | X | | | 1 | H |
| 259 | X | | | | 1 | H |
| 262 | X | | | | 1 | H |
| 263 | X | | | X | 1 | H |
| 264 | | X | | | 1 | H |
| 266 | X | | | | 1 | O |
| 307 | X | | | X | 1 | n/a |
| 291 | | | X | X | 1 | O |
| 293 | | X | | X | 3 | O |
| 297 | | | X | | 1 | O |
| 298 | | X | | | 1 | O |
| 299 | | X | | | 1 | O |
| 300 | | | X | X | 1 | O |
| 301 | | | X | | 1 | n/a |
| 302 | | | X | | 1 | O |
| 303 | | | X | | 2 | O |
| 304 | | | X | | 1 | O |
| 311 | | | X | | 1 | H |
| 314 | | | X | | 2 | O |
| 315 | | | X | | 1 | O |
| 324 | | | X | | 2 | O |

Table 2 summarizes duration of treatment of patients either until further intervention or end of study. Majority of patients were on Clinical Study I or Clinical Study II treatment for at least 12 months, and of which, over 84% were on treatment for at least 6 months.

TABLE 2
Duration of ultrasound treatment

| <3 months | 3-5 months | 6-11 months | 12 to 24 months | >24 months |
|---|---|---|---|---|
| 2 | 8 | 15 | 22 | 19 |

### 9.1.2 Results

A total of 30 treated eyes and 28 non-treated (contralateral) eyes of patients (from both studies) whose records were verifiable before and after at least one ultrasound treatment were evaluated. At follow-up visit after the last treatment (e.g., 12 months after ultrasound treatment), patients were evaluated for (1) thickness of RNFL, and (2) cup to disc ratio measured by OCT or HRT.

9.1.2.1 RNFL thickness

Out of the 30 treated eyes, the thickness of the RNFL of 17 eyes (56.67%) either remained the same or increased in thickness. Of the 28 non-treated (contralateral) eyes, the RNFL of 11 eyes (39.29%) either remained the same or increased in thickness. The results were shown in Table 3. Tx RNFL (treated RNFL) at baseline indicates RNFL of treated eye measured at baseline (pre-treatment). Cx RNFL (contralateral control RNFL) at baseline indicates RNFL of contralateral untreated eye measured at baseline (pre-treatment). Tx RNFL at follow-up indicates RNFL of treated eye measured at first follow-up after at least one ultrasound treatment (post-treatment). Cx RNFL at follow-up indicates RNFL of contralateral untreated eye measured at first follow-up after at least one ultrasound treatment (post-treatment). Table 3 shows the effect of the ultrasound treatment on the RNFL in both the treated eye (Tx) and the contralateral eye (Cx) and the length of follow up of these patients.

TABLE 3
RNFL change from baseline and follow-up evaluation after at least one treatment

| | | | | | Baseline | Tx | | Cx | |
|---|---|---|---|---|---|---|---|---|---|
| | Tx | Cx | | Ultrasound | to | RNFL | % | RNFL | % |
| | RNFL | RNFL | # of Ultrasound | Treatment | follow- | at | Change | at | Change |
| Patient | at | at | Treatment | to follow- | up | follow- | in Tx | follow- | in Cx |
| # | baseline | baseline | (UT) | up (days) | (days) | up | eye | up | eye |
| 202 | 0.114 | 0.196 | 3 | 512 | 807 | 0.16 | 40.35% | 0.182 | −7.14% |
| 216 | 0.009 | 0.084 | 3 | 370 | 819 | 0.05 | 455.56% | 0.11 | 30.95% |
| 229 | 68 | 73 | 1 | 159 | 404 | 69 | 1.47% | 71 | −2.74% |
| 236 | 0.06 | n/a | 1 | 230 | 394 | 0.078 | 30.00% | n/a | n/a |
| 237 | 0.192 | 0.134 | 1 | 299 | 480 | 0.181 | −5.73% | 0.13 | −2.99% |
| 243 | 0.099 | 0.141 | 2 | 270 | 724 | 0.089 | −10.10% | 0.151 | 7.09% |
| 249 | 69 | 79 | 1 | 552 | 910 | 69 | 0.00% | 77 | −2.53% |
| 251 | 0.135 | 0.087 | 1 | 601 | 768 | 0.18 | 33.33% | 0.046 | −47.13% |
| 252 | 0.073 | 0.177 | 1 | 253 | 421 | 0.11 | 50.68% | 0.216 | 22.03% |
| 254 | 0.182 | 0.188 | 1 | 161 | 543 | 0.2 | 9.89% | 0.192 | 2.13% |
| 259 | 0.26 | 0.171 | 2 | 189 | 455 | 0.229 | −11.92% | 0.173 | 1.17% |
| 262 | 62 | 76 | 1 | 235 | 387 | 62 | 0.00% | 79 | 3.95% |
| 263 | 0.192 | 0.233 | 1 | 537 | 727 | 0.252 | 31.25% | 0.217 | −6.87% |
| 264 | 0.142 | n/a | 1 | 113 | 353 | 0.17 | 19.72% | n/a | n/a |
| 266 | 68 | 82 | 1 | 329 | 349 | 70 | 2.94% | 81 | −1.22% |
| 291 | 50 | 58 | 1 | 218 | 357 | 52 | 4.00% | 61 | 5.17% |
| 293 | 74 | 87 | 3 | 250 | 404 | 68 | −8.11% | 80 | −8.05% |
| 297 | 62 | 79 | 1 | 247 | 411 | 55 | −11.29% | 70 | −11.39% |
| 298 | 73 | 74 | 1 | 329 | 377 | 73 | 0.00% | 76 | 2.70% |
| 299 | 80 | 85 | 1 | 230 | 508 | 83 | 3.75% | 82 | −3.53% |
| 300 | 85 | 86 | 1 | 97 | 461 | 81 | −4.71% | 84 | −2.33% |
| 301 | 93 | 87 | 1 | 253 | 35 | 91 | −2.15% | 90 | 3.45% |
| 302 | 93 | 97 | 1 | 348 | 360 | 94 | 1.08% | 96 | −1.03% |
| 303 | 82 | 82 | 2 | 160 | 462 | 80 | −2.44% | 73 | −10.98% |
| 304 | 83 | 94 | 1 | 119 | 386 | 75 | −9.64% | 73 | −22.34% |
| 307 | 74 | 69 | 1 | 264 | 408 | 72 | −2.70% | 67 | −2.90% |
| 311 | 80 | 82 | 1 | 271 | 454 | 77 | −3.75% | 82 | 0.00% |
| 314 | 65 | 68 | 2 | 265 | 1134 | 59 | −9.23% | 60 | −11.76% |
| 315 | 52 | 57 | 1 | 336 | 411 | 49 | −5.77% | 56 | −1.75% |
| 324 | 69 | 69 | 1 | 97 | 342 | 72 | 4.35% | 72 | 4.35% |
| | | | | | Samples | | 30 | | 28 |
| | | | | | > = 0 | | 17 | | 11 |
| | | | | | % > = 0 | | 56.67% | | 39.29% |

*Cx: contralateral control
Tx: ultrasound treatment

### 9.1.2.1.1 Ultrasound Treatment Increased RNFL Thickness in Treatment Naïve Patients

Ultrasound treatment increased RNFL thickness in treatment naïve patients. Referring to Tables 1 and 3, Patient 216 who was treatment naïve (was off medication for at least 12 months) received three treatments. RNFL was measured at baseline and at a follow-up visit. The thickness of RNFL in the treated eye increased by about 455.56% and the thickness of RNFL in the contralateral eye increased by about 30.95% as measured at 370 days (over 12 months) after the final treatment as compared to RNFL thickness at baseline. As used herein, the term “final treatment” refers to the last ultrasound treatment given to the patient at the end of the study or at the latest verifiable treatment.

Patient 252 was treatment naïve and received one treatment. The thickness of RNFL in the treated eye at the last follow-up increased by about 50.68% and the thickness of RNFL in the contralateral eye increased by about 22.03% as measured at 253 days (over 8 months) at a follow-up visit after the final treatment as compared to baseline thickness.

Patient 259 was treatment naïve and received one treatment. The thickness of RNFL in the treated eye at the last follow-up reduced by about 11.92% and the thickness of RNFL in the contralateral eye increased by about 1.17% as measured at 189 days (over 6 months) at a follow-up visit after the final treatment as compared to baseline thickness.

Patient 262 was treatment naïve and received one treatment. The thickness of RNFL in the treated eye at the last follow-up maintained unchanged and the thickness of RNFL in the contralateral eye increased by about 3.95% as measured at 235 days (over 7 months) at a follow-up visit after the final treatment as compared to baseline thickness.

Patient 263 was treatment naïve and received one treatment. The thickness of RNFL in the treated eye at the last follow-up increased by 31.25% and the thickness of RNFL in the contralateral eye decreased by about 6.87% as measured at 537 days (about 18 months) at a follow-up visit after the final treatment as compared to baseline thickness.

Patient 266 was treatment naïve and received one treatment. The thickness of RNFL in the treated eye at the last follow-up increased by 2.94% and the thickness of RNFL in the contralateral eye decreased by about 1.22% as measured at 329 days (about 11 months) at a follow-up visit after the final treatment as compared to baseline thickness.

Patient 307 was treatment naïve and received one treatment. The thickness of RNFL in the treated eye at the last follow-up decreased by 2.7% and the thickness of RNFL in the contralateral eye decreased by about 2.9% as measured at 264 days (over 8 months) at a follow-up visit after the final treatment as compared to baseline thickness.

The data demonstrate that ultrasound treatment as a monotherapy is effective in increasing RNFL thickness and/or reducing or preventing RNFL thinning, and/or reducing IOP to a normal or healthy level in patient population who had never been treated for glaucoma or who had been off pharmaceutical agents for at least 6 months. Further, the data demonstrated that ultrasound treatment was safe for repeated treatments, which further augmented and/or sustained the efficacy.

9.1.2.1.2 Ultrasound Treatment Increased RNFL Thickness in Patients Who had Priorly been Treated with a Pharmacological Agent

Ultrasound treatment increased RNFL thickness in patients who had priorly been treated with a pharmacological agent. Patients were on one or more pharmaceutical agents and were taken off the drug at least 30 days (washout) before administering the ultrasound treatment.

Referring to Tables 1 and 3, Patient 202 received three treatments. RNFL was measured at baseline and at a follow-up visit. The thickness of RNFL in the treated eye at the last or most recent follow up increased by about 40.35% and the thickness of RNFL in the contralateral eye decreased by about 7.14% as measured at 512 days (over 17 months) after the final treatment (e.g., at last follow-up visit) as compared to baseline thickness. Patient 229 received one treatment. The thickness of RNFL in the treated eye at the last or most recent follow up increased by about 1.47% and the thickness of RNFL in the contralateral eye decreased by about 2.74% as measured at 159 days (over 5 months) after the final treatment as compared to baseline thickness.

Patient 236 received one treatment. The thickness of RNFL in the treated eye at the last or most recent follow up increased by about 30% and the thickness of RNFL in the contralateral eye maintained unchanged as measured at 230 days (over 7 months) after the final treatment.

Patient 237 received one treatment. The thickness of RNFL in the treated eye at the last or most recent follow up decreased by about 5.73% and the thickness of RNFL in the contralateral eye decreased by 2.99% as measured at 230 days (over 7 months) after the final treatment as compared to baseline thickness.

Patient 243 received two treatments. The thickness of RNFL in the treated eye at the last or most recent follow up decreased by about 10.1% and the thickness of RNFL in the contralateral eye increased by 7.09% as measured at 270 days (9 months) after the final treatment as compared to baseline thickness.

Patient 249 received one treatment. The thickness of RNFL in the treated eye at the last or most recent follow up maintained unchanged and the thickness of RNFL in the contralateral eye decreased by 2.53% as measured at 552 days (over 18 months) after the final treatment as compared to baseline thickness.

Patient 251 received one treatment. The thickness of RNFL in the treated eye at the last or most recent follow up increased 33.33% and the thickness of RNFL in the contralateral eye decreased by 47.13% as measured at 601 days (over 20 months) after the final treatment as compared to baseline thickness.

Patient 254 received one treatment. The thickness of RNFL in the treated eye at the first follow-up increased by about 9.89% and the thickness of RNFL in the contralateral eye increased by about 2.13% as measured at 161 days (over 5 months) after the final treatment as compared to baseline thickness.

Patient 264 received one treatment. The thickness of RNFL in the treated eye at the first follow-up increased by 19.72% and the thickness of RNFL in the contralateral eye maintained unchanged as measured at 113 days (over 3 months) after the final treatment as compared to baseline thickness.

Patient 293 received three treatments. The thickness of RNFL in the treated eye at the first follow-up decreased by 8.11% and the thickness of RNFL in the contralateral eye decreased by 8.05% as measured at 250 days (over 8 months) after the final treatment as compared to baseline thickness.

Patient 298 received one treatment. The thickness of RNFL in the treated eye at the first follow-up maintained unchanged and the thickness of RNFL in the contralateral eye increased by 2.7% as measured at 329 days (over 10 months) after the final treatment as compared to baseline thickness.

Patient 299 received one treatment. The thickness of RNFL in the treated eye at the first follow-up increased by 3.75% and the thickness of RNFL in the contralateral eye decreased by 3.53% as measured at 230 days (over 7 months) after the final treatment as compared to baseline thickness.

The data demonstrate that ultrasound treatment as a monotherapy is effective in increasing RNFL thickness and/or reducing or preventing RNFL thinning, and/or reducing IOP to an normal or healthy level in patient population who had been treated for glaucoma but progressed following the treatment.

### 9.1.2.1.3 Ultrasound Treatment Increased RNFL Thickness in Patients Who are on a Pharmacological Agent and Reduced the Need for Pharmaceutical Agent

Ultrasound treatment increased RNFL thickness in patients who are on a pharmacological agent, ultrasound treatment was used as a complementary or ancillary treatment to existing pharmacological agents that the patient was receiving throughout the study. Patients may be having one or more pharmacological agents (e.g., not more than 6, 5, 4, 3, or 2). Exemplary pharmaceutical agents include from β-blockers, carbonic anhydrase inhibitors, prostaglandin analog, a2-adrenergic agonists, parasympathomimetic drugs, and/or combinations thereof. Patients were monitored for their IOP change between every visit. At the follow-up visit, if a patient's IOP decreased as compared to baseline or as compared to health IOP (e.g., 22 mmHg to 35 mmHg), the patient was taken off from a pharmaceutical agent. One agent was taken off at a time. If the patient's IOP maintained or increased as compared to the baseline level, the patient was given an additional ultrasound treatment.

Referring to Tables 1 and 3, Patient 291 was on two medications, had an IOP of 14 mmHg and received one treatment. RNFL was measured at baseline and at a follow-up visit. The thickness of RNFL in the treated eye at the last or most recent follow up increased by about 40.35% and the thickness of RNFL in the contralateral eye decreased by about 7.14% as measured at 512 days (over 17 months) after the final treatment (e.g., at last follow-up visit) as compared to baseline thickness. Patient 291 was taken off from all medications (zero medication) and had an IOP of 13 mmHg at the completion of ultrasound treatment.

Patient 297 was on one medication, had an IOP of 21 mmHg, and received one treatment. The thickness of RNFL in the treated eye at the last or most recent follow up decreased by 11.29% and the thickness of RNFL in the contralateral eye decreased by about 11.39% as measured at 247 days (over 8 months) after the final treatment (e.g., at last follow-up visit) as compared to baseline thickness. Patient 297 maintained on one medication and had an IOP of 13.75 mmHg at the completion of ultrasound treatment.

Patient 300 was on one medication, had an IOP of 13 mmHg and received one treatment. The thickness of RNFL in the treated eye at the last or most recent follow up decreased by 4.71% and the thickness of RNFL in the contralateral eye decreased by about 2.33% as measured at 97 days (over 3 months) after the final treatment (e.g., at last follow-up visit) as compared to baseline thickness. Patient 300 maintained on one medication and had an IOP of 10.25 mmHg at the completion of ultrasound treatment.

Patient 301 was on three medications, had an IOP of 18 mmHg and received one treatment. The thickness of RNFL in the treated eye at the last or most recent follow up decreased by 2.15% and the thickness of RNFL in the contralateral eye increased by about 3.45% as measured at 253 days (over 8 months) after the final treatment (e.g., at last follow-up visit) as compared to baseline thickness. Patient 301 was on one medication, a reduction from three to one, and had an IOP of 16.75 mmHg at the completion of ultrasound treatment.

Patient 302 was on one medication, had an IOP of 26 mmHg and received one treatment. The thickness of RNFL in the treated eye at the last or most recent follow up increased by 1.08% and the thickness of RNFL in the contralateral eye decreased by about 1.03% as measured at 348 days (over 11 months) after the final treatment (e.g., at last follow-up visit) as compared to baseline thickness. Patient 302 maintained on one medication and had an IOP of 18.5 mmHg at the completion of ultrasound treatment.

Patient 303 was on one medication, had an IOP of 16 mmHg and received two treatments. The thickness of RNFL in the treated eye at the last or most recent follow up decreased by 2.44% and the thickness of RNFL in the contralateral eye decreased by about 10.98% as measured at 160 days (over 5 months) after the final treatment (e.g., at last follow-up visit) as compared to baseline thickness. Patient 303 maintained on one medication and had an IOP of 13 mmHg at the completion of ultrasound treatment.

Patient 304 was one two medications, had an IOP of 15 mmHg and received one treatment. The thickness of RNFL in the treated eye at the last or most recent follow up decreased by 9.64% and the thickness of RNFL in the contralateral eye decreased by 22.34% as measured at 119 days (about 4 months) after the final treatment (e.g., at last follow-up visit) as compared to baseline thickness. Patient 304 was off on all medications (zero medication) and had an IOP of 13 mmHg at the completion of ultrasound treatment.

Patient 311 was on two medications, had an IOP of 18 mmHg and received one treatment. The thickness of RNFL in the treated eye at the last or most recent follow up decreased by 3.75% and the thickness of RNFL in the contralateral eye maintained unchanged as measured at 271 days (over 9 months) after the final treatment (e.g., at last follow-up visit) as compared to baseline thickness. Patient 311 maintained two medications and had an IOP of 10 mmHg at the completion of ultrasound treatment.

Patient 314 was on five medications, had an IOP of 17 mmHg and received two treatments. The thickness of RNFL in the treated eye at the last or most recent follow up decreased by 9.23% and the thickness of RNFL in the contralateral eye decreased by 11.76% as measured at 265 days (over 8 months) after the final treatment (e.g., at last follow-up visit) as compared to baseline thickness. Patient 314 maintained on three medications, a reduction from five to three, and had an IOP of 22.5 mmHg at the completion of ultrasound treatment.

Patient 315 was on three medications, had an IOP of 11 mmHg and received one treatment. The thickness of RNFL in the treated eye at the last or most recent follow up decreased by 5.77% and the thickness of RNFL in the contralateral eye decreased by 1.75% as measured at 336 days (over 11 months) after the final treatment (e.g., at last follow-up visit) as compared to baseline thickness. Patient 315 maintained three medications and had an IOP of 11.5 mmHg at the completion of ultrasound treatment.

Patient 324 was on three medications, had an IOP of 11 mmHg and received two treatments. The thickness of RNFL in the treated eye at the last or most recent follow up increased by 4.35% and the thickness of RNFL in the contralateral eye increased by 4.35% as measured at 336 days (over 11 months) after the final treatment (e.g., at last follow-up visit) as compared to baseline thickness. Patient 324 was off all medications, a reduction from three to zero, and had an IOP of 12 mmHg at the completion of ultrasound treatment.

The data demonstrate that ultrasound treatment was safe to be used as a complementary or ancillary treatment and is effective in increasing RNFL thickness and/or reducing or preventing RNFL thinning in a patient population who were receiving pharmaceutical agents. The data demonstrate that ultrasound treatment could potentiate the effectiveness pharmaceutical agents in lowering IOP, increasing RNFL thickness, and/or preventing or reducing RNFL thinning. Ultrasound treatment reduced the number of medications needed to maintain a patient's IOP at a normal or acceptable level. Additionally, ultrasound treatment could provide additive effect to pharmaceutical agents in treating patients who have elevated IOP and/or increased RNFL thinning, and/or reducing.

### 9.1.2.1.4 Ultrasound Treatment Increased RNFL Thickness in Patients Having Normotensive Glaucoma

Ultrasound treatment increased RNFL thickness in patients having normotensive glaucoma, also known as normal-tension glaucoma. Normotensive glaucoma is a specific type of glaucoma where the damage occurs to the optic nerve without elevated intraocular pressure (IOP). In this condition, the IOP falls within the normal range (e.g., between 10 to 21 mmHg) and yet the optic nerve suffers damage leading to the symptomatic vision loss characteristic of glaucoma. Normotensive glaucoma can be diagnosed by ophthalmoscopy, visual field testing, and/or optical coherence tomography (OCT). Due to the absence of elevated IOP, lowering IOP is not the primary focus for patients who have normotensive glaucoma. Treatments for normotensive glaucoma are limited, and these can include neuroprotective medications, laser procedures, and/or lifestyle modifications.

Referring to Tables 1 and 3, Patient 216, Patient 243, Patient 251, Patient 263, Patient 291, Patient 293, Patient 300, and Patient 307 were diagnosed with normotensive glaucoma. Among which, Patient 216, Patient 251, Patient 263, and Patient 291 had increased RNFL by at least 4% (about 4% to about 455.56%) as compared to baseline.

The data demonstrate that ultrasound treatment was effective in increasing RNFL thickness and/or reducing or preventing RNFL thinning in a patient population who were diagnosed with normotensive glaucoma and had limited treatment options.

The thickening of the RNFL was not associated with edema as no decrease in visual acuity or vision field was found in all patients showing increased RNFL. In all patients who had increased RNFL, the increased thickness of RNFL was persistent and sustained over at least 1 month (e.g., 5, 8, 17 months as described above). Without being bound by a theory, the increase in thickness of the contralateral eyes may be attributed to a trigger of systemic cytokines produced from a corresponding treated eye of a subject in response to ultrasound treatment. The data demonstrated that the method and device were effective in reducing progression of RNFL thinning and/or promoting growth of cells in the RNFL over at least 30 days (1 month).

### 9.1.2.2 Factoring in the Median Loss of RNFL Per Year

Factoring in the median loss of RNFL per year showed more significant improvement of RNFL thickness. Studies have demonstrated that when the baseline RNFL thickness was used as a reference (100%), the proportional loss of RNFL thickness ranged from 1.8% to 12.4% per year with a median loss of 3.8% per year. See e.g., Leung et al. Investigative Ophthalmology & Visual Science, Vol. 51, 217-222 (2010), which is incorporated by reference herein in its entirety.

When the data of Table 3 factor in the median loss of RNFL per year of approximately 3.8% in post-hoc analysis, the results of ultrasound treatment were even more significant. It is surprising that ultrasound treatment not only prevented or reduced RNFL thinning, but also significantly increased RNFL thickness in glaucomatous eyes or eyes with elevated IOP.

Expected RNFL thickness was calculated as the following formula:

Expected RNFL thickness=Baseline “pre-ultrasound treatment”−((3.8%*Baseline RNFL)*(# of days from ultrasound treatment to first follow-up/365)).

Out of the 30 treated eyes, the thickness of the RNFL of 23 eyes (76.67%) either remained the same or increased in thickness. Of the 28 non-treated (contralateral) eyes, the RNFL of 23 eyes (82.14%) either remained the same or increased in thickness. The data demonstrate that ultrasound treatment not only was effective in reducing IOP and prevention of RNFL thinning, but also significantly increased RNFL.

Table 4 summarizes RNFL change from baseline and follow-up evaluation after at least one treatment factoring in the median loss of RNFL per year of approximately 3.8%. Tx RNFL at baseline indicates RNFL of treated eye measured at baseline (pre-treatment). Cx RNFL at baseline indicates RNFL of contralateral untreated eye measured at baseline (pre-treatment). Tx RNFL at follow-up indicates RNFL of treated eye measured at first follow-up after at least one ultrasound treatment (post-treatment). Cx RNFL at follow-up indicates RNFL of contralateral untreated eye measured at first follow-up after at least one ultrasound treatment (post-treatment). Table 4. RNFL change from baseline and follow-up evaluation after at least one treatment factoring in the median loss of RNFL per year of approximately 3.8%.

TABLE 4
RNFL change from baseline and follow-up evaluation after at least one treatment factoring in
the median loss of RNFL per year of approximately 3.8%.

| | | | | | | | % | | % |
|---|---|---|---|---|---|---|---|---|---|
| | | | | | Baseline | | Change | | Change |
| | Tx | Cx | | Ultrasound | to | Tx | in Tx | Cx | is Cx |
| | RNFL | RNFL | # of | treatment | follow | RNFL at | with | RNFL at | with |
| Patient | at | at | ultrasound | to follow | up | follow | 3.8% | follow | 3.8% |
| # | baseline | baseline | treatment | up (days) | (days) | up | loss/yr | up | loss/yr |
| 202 | 0.114 | 0.196 | 3 | 512 | 807 | 0.16 | 53.22% | 0.182 | 1.37% |
| 216 | 0.009 | 0.084 | 3 | 370 | 819 | 0.05 | 507.34% | 0.11 | 43.16% |
| 229 | 68 | 73 | 1 | 159 | 404 | 69 | 5.93% | 71 | 1.53% |
| 236 | 0.06 | n/a | 1 | 230 | 394 | 0.078 | 35.56% | n/a | n/a |
| 237 | 0.192 | 0.134 | 1 | 299 | 480 | 0.181 | −0.77% | 0.13 | 2.12% |
| 243 | 0.099 | 0.141 | 2 | 270 | 724 | 0.089 | −2.77% | 0.151 | 15.82% |
| 249 | 69 | 79 | 1 | 552 | 910 | 69 | 10.47% | 77 | 7.67% |
| 251 | 0.135 | 0.087 | 1 | 601 | 768 | 0.18 | 44.92% | 0.046 | −42.53% |
| 252 | 0.073 | 0.177 | 1 | 253 | 421 | 0.11 | 57.59% | 0.216 | 27.63% |
| 254 | 0.182 | 0.188 | 1 | 161 | 543 | 0.2 | 16.47% | 0.192 | 8.25% |
| 259 | 0.26 | 0.171 | 2 | 189 | 455 | 0.229 | −7.54% | 0.173 | 6.20% |
| 262 | 62 | 76 | 1 | 235 | 387 | 62 | 4.20% | 79 | 8.31% |
| 263 | 0.192 | 0.233 | 1 | 537 | 727 | 0.252 | 42.00% | 0.217 | 0.76% |
| 264 | 0.142 | n/a | 1 | 113 | 353 | 0.17 | 24.29% | n/a | n/a |
| 266 | 68 | 82 | 1 | 329 | 349 | 70 | 6.82% | 81 | 2.50% |
| 291 | 50 | 58 | 1 | 218 | 357 | 52 | 8.01% | 61 | 9.23% |
| 293 | 74 | 87 | 3 | 250 | 404 | 68 | −4.07% | 80 | −4.01% |
| 297 | 62 | 79 | 1 | 247 | 411 | 55 | −7.32% | 70 | −7.43% |
| 298 | 73 | 74 | 1 | 329 | 377 | 73 | 4.09% | 76 | 6.90% |
| 299 | 80 | 85 | 1 | 230 | 508 | 83 | 9.54% | 82 | 1.86% |
| 300 | 85 | 86 | 1 | 97 | 461 | 81 | 0.10% | 84 | 2.60% |
| 302 | 93 | 97 | 1 | 348 | 360 | 94 | 5.01% | 96 | 2.82% |
| 303 | 82 | 82 | 2 | 160 | 462 | 80 | 2.49% | 73 | −6.48% |
| 304 | 83 | 94 | 1 | 119 | 386 | 75 | −5.86% | 73 | −19.09% |
| 311 | 80 | 82 | 1 | 271 | 454 | 77 | 1.03% | 82 | 4.96% |
| 314 | 65 | 68 | 2 | 265 | 1134 | 59 | 2.92% | 60 | 0.05% |
| 315 | 52 | 57 | 1 | 336 | 411 | 49 | −1.56% | 56 | 2.64% |
| 324 | 69 | 69 | 1 | 97 | 342 | 72 | 8.20% | 72 | 8.20% |
| | | | | | Samples | | 30 | | 28 |
| | | | | | > = 0 | | 23 | | 23 |
| | | | | | % > = 0 | | 76.67% | | 82.14% |

RNFL thickness tend to naturally decrease with age, even in healthy individuals. Referring to Table 4, after taking into account the median loss of RNFL per year associated with aging, the effectiveness of ultrasound treatment was significant in patients who appeared to have no change or decreased RNFL. It is noted that while Patient 262 (treatment naïve) and Patient 249 (washout) both appeared to have change of RNFL. Normalizing the data with the median RNFL per year showed that ultrasound treatment increased RNFL thickness by 4.2% for Patient 262 and by 10.47% for Patient 249, as compared to baseline thickness. Normalizing showed that Patient 302 and Patient 324 had increased RNFL by 5.01% and 8.2%, respectively, as compared to baseline thickness.

### 9.1.2.3 Cup to Disc Ratio

The disc represents the head of the optic nerve. The cup represents atrophy within the center of the optic nerve. The larger the cup, the more atrophy of the optic nerve. The Cup/Disc (C/D) ratio is a parameter followed over time to evaluate the adequacy of glaucoma treatment. Along with a decreasing RNFL, an increasing C/D ratio is a significant indicator of inadequate glaucoma treatment. On the other hand, no change in the C/D ratio is one indicator of adequate treatment.

Out of the 30 treated eyes, the cup to disc ratio of 17 eyes or about 56.67% patients either remained the same or decreased. Of the 28 non-treated (contralateral) eyes, the cup to disc ratio of 9 eyes or about 32.14% patients either remained the same or decreased. Cup to disc ratio was measured using OCT or HRT. The results are shown in Table 4. Tx C/D at baseline indicates cup to disc ratio of treated eye measured at baseline (pre-treatment). Cx C/D at baseline indicates cup to disc ratio of contralateral untreated eye measured at baseline (pre-treatment). Tx C/D at follow-up indicates cup to disc ratio of treated eye measured at first follow-up after at least one ultrasound treatment (post-treatment). Cx C/D at follow-up indicates cup to disc of contralateral untreated eye measured at first follow-up after at least one ultrasound treatment (post-treatment). For example, Patient 216 received three treatments. The cup to disc ratio in the treated eye decreased by about 4.25% and the cup to disc ratio in the contralateral eye increased by about 5.18% as measured at 370 days after the final treatment. Patient 252 received one treatment. The cup to disc ratio in the treated eye decreased by about 5.82% and the cup to disc ratio in the contralateral eye decreased about 19.68% as measured at 253 days after the final treatment (follow-up visit). Patient 202 received three treatments. The cup to disc ratio in the treated eye was reduced by about 19.57% and the cup to disc ratio in the contralateral eye increased by about 0.94% as measured at 512 days after the final treatment (follow-up visit). Patient 254 received one treatment. The cup to disc ratio in the treated eye decreased by about 2.29% and the cup to disc ratio in the contralateral eye increased by about 0.47% as measured at 161 days after the final treatment (follow-up visit). Table 5 shows Cup to Disc ratio change from baseline and follow-up evaluation after at least one treatment.

TABLE 5
Cup to Disc ratio change from baseline and follow-up evaluation after at least
one treatment

| | | | Number of | Days | | | | | |
|---|---|---|---|---|---|---|---|---|---|
| | Tx C/D | Cx C/D | ultrasound | from UT | Baseline | Tx C/D | % | Cx C/D | % |
| Patient | at | at | treatment | to follow- | to follow- | at follow- | Change in | at follow- | Change in |
| # | baseline | baseline | (UT) | up | up: days | up | Tx eye | up | Cx eye |
| 202 | 0.414 | 0.424 | 3 | 512 | 807 | 0.333 | −19.57% | 0.428 | 0.94% |
| 216 | 0.847 | 0.598 | 3 | 370 | 819 | 0.811 | −4.25% | 0.629 | 5.18% |
| 229 | 0.76 | 0.8 | 1 | 159 | 404 | 0.76 | 0.00% | 0.8 | 0.00% |
| 236 | 0.592 | | 1 | 230 | 394 | 0.503 | −15.03% | | |
| 237 | 0.66 | 0.727 | 1 | 299 | 480 | 0.67 | 1.52% | 0.737 | 1.38% |
| 243 | 0.736 | 0.734 | 2 | 270 | 724 | 0.791 | 7.47% | 0.731 | −0.41% |
| 249 | 0.65 | 0.61 | 1 | 552 | 910 | 0.73 | 12.31% | 0.63 | 3.28% |
| 251 | 0.726 | 0.681 | 1 | 601 | 768 | 0.709 | −2.34% | 0.701 | 2.94% |
| 252 | 0.67 | 0.31 | 1 | 253 | 421 | 0.631 | −5.82% | 0.249 | −19.68% |
| 254 | 0.48 | 0.424 | 1 | 161 | 543 | 0.469 | −2.29% | 0.426 | 0.47% |
| 259 | 0.402 | 0.551 | 2 | 189 | 455 | 0.411 | 2.24% | 0.577 | 4.72% |
| 262 | 0.62 | 0.59 | 1 | 235 | 387 | 0.63 | 1.61% | 0.62 | 5.08% |
| 263 | 0.48 | 0.612 | 1 | 537 | 727 | 0.457 | −4.79% | 0.589 | −3.76% |
| 264 | 0.664 | | 1 | 113 | 353 | 0.653 | −1.66% | | |
| 266 | 0.75 | 0.71 | 1 | 329 | 349 | 0.73 | −2.67% | 0.73 | 2.82% |
| 291 | 0.77 | 0.82 | 1 | 218 | 357 | 0.79 | 2.60% | 0.84 | 2.44% |
| 293 | 0.73 | 0.43 | 3 | 250 | 404 | 0.76 | 4.11% | 0.44 | 2.33% |
| 297 | 0.58 | 0.42 | 1 | 247 | 411 | 0.61 | 5.17% | 0.41 | −2.38% |
| 298 | 0.32 | 0.09 | 1 | 329 | 377 | 0.3 | −6.25% | 0.08 | −11.11% |
| 299 | 0.79 | 0.67 | 1 | 230 | 508 | 0.72 | −8.86% | 0.59 | −11.94% |
| 300 | 0.75 | 0.72 | 1 | 97 | 461 | 0.76 | 1.33% | 0.75 | 4.17% |
| 301 | 0.18 | 0.23 | 1 | 253 | 351 | 0.25 | 38.89% | 0.28 | 21.74% |
| 302 | 0.43 | 0.46 | 1 | 348 | 360 | 0.47 | 9.30% | 0.45 | −2.17% |
| 303 | 0.8 | 0.76 | 2 | 160 | 462 | 0.79 | −1.25% | 0.78 | 2.63% |
| 304 | 0.7 | 0.6 | 1 | 119 | 386 | 0.7 | 0.00% | 0.63 | 5.00% |
| 307 | 0.71 | 0.7 | 1 | 264 | 408 | 0.71 | 0.00% | 0.68 | −2.86% |
| 311 | 0.75 | 0.73 | 1 | 271 | 454 | 0.75 | 0.00% | 0.69 | −5.48% |
| 314 | 0.76 | 0.74 | 2 | 265 | 1134 | 0.8 | 5.26% | 0.78 | 5.41% |
| 315 | 0.84 | 0.86 | 1 | 336 | 411 | 0.84 | 0.00% | 0.88 | 2.33% |
| 324 | 0.63 | 0.62 | 1 | 97 | 342 | 0.65 | 3.17% | 0.64 | 3.23% |
| | | | | | Samples | | 30 | | 28 |
| | | | | | <= 0 | | 17 | | 9 |
| | | | | | % < = 0 | | 56.67% | | 32.14% |

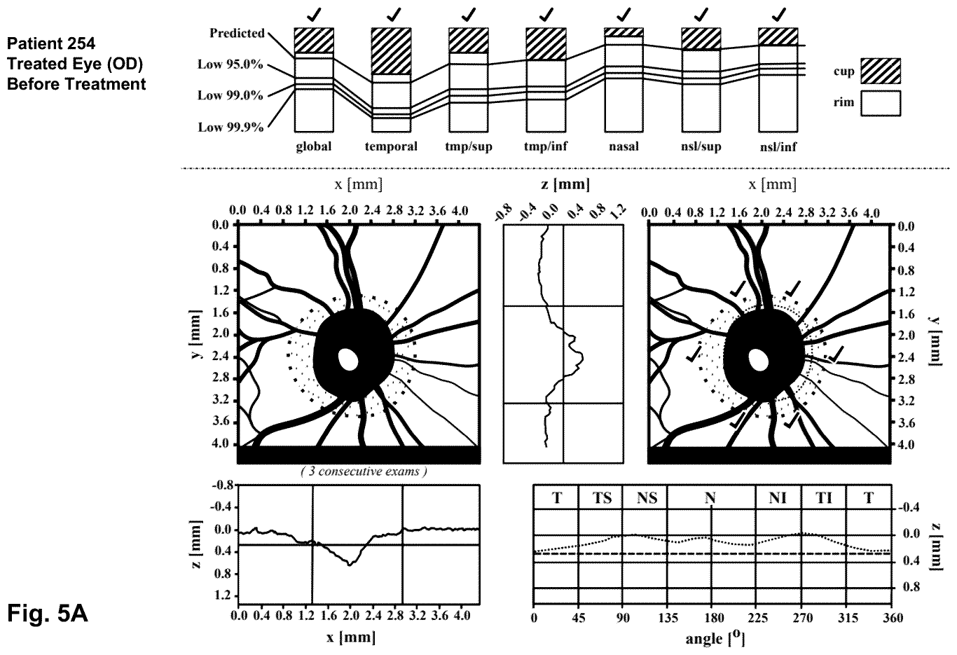

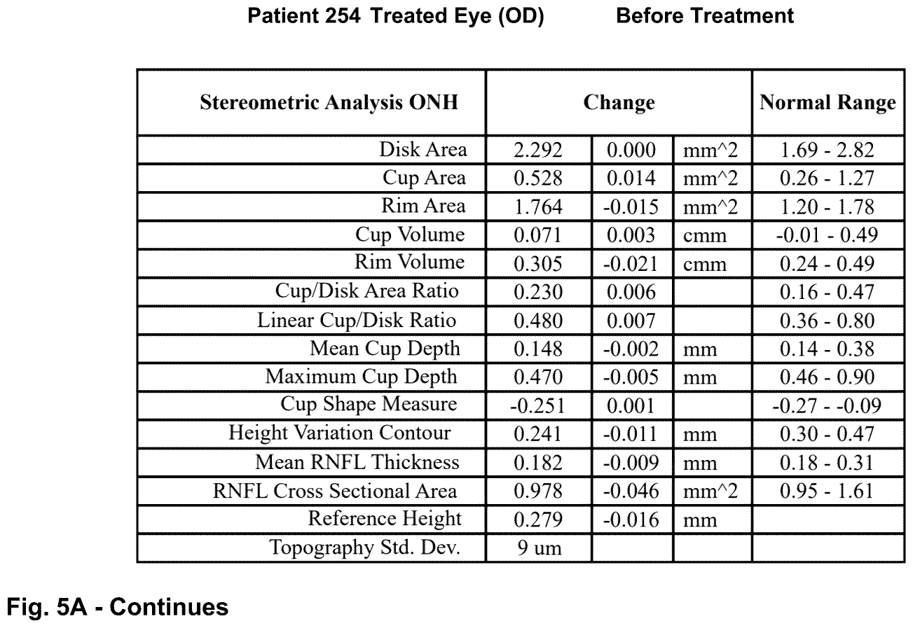

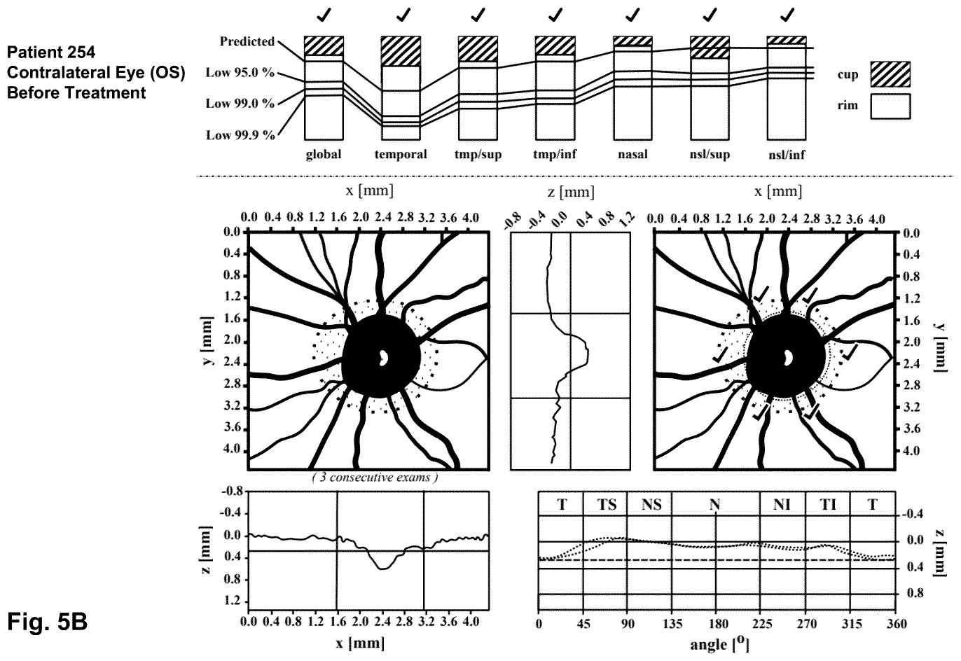

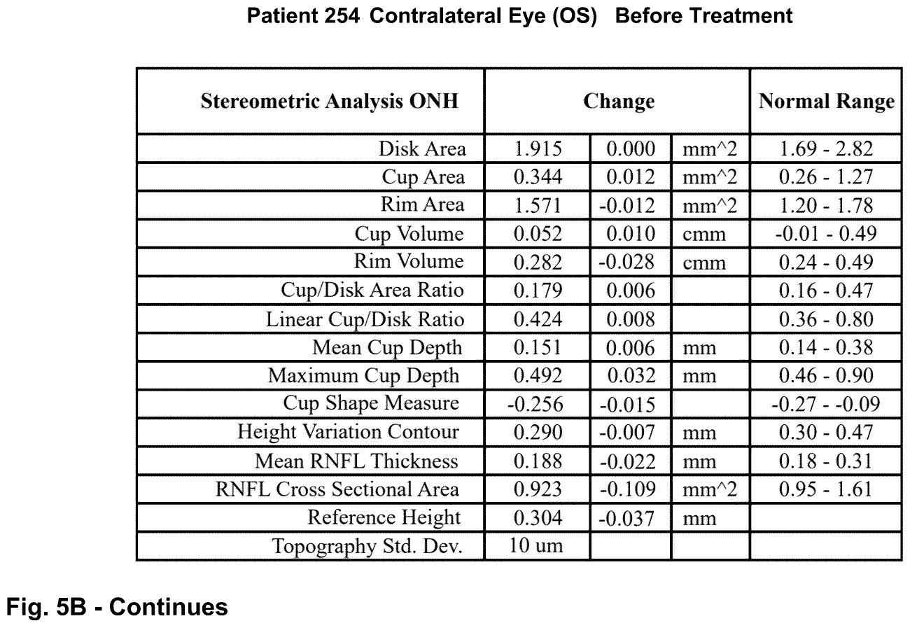

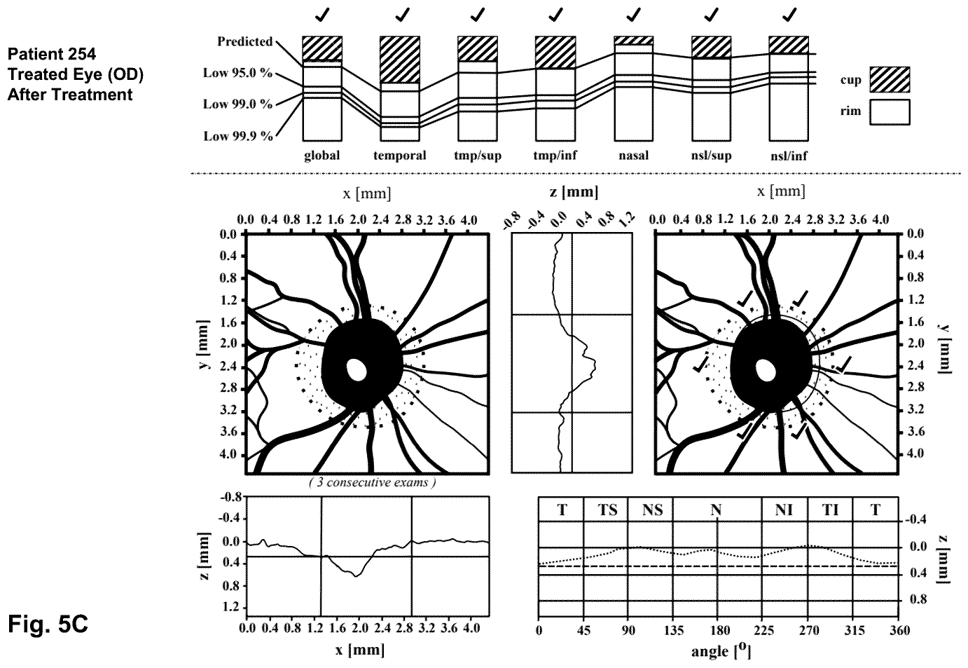

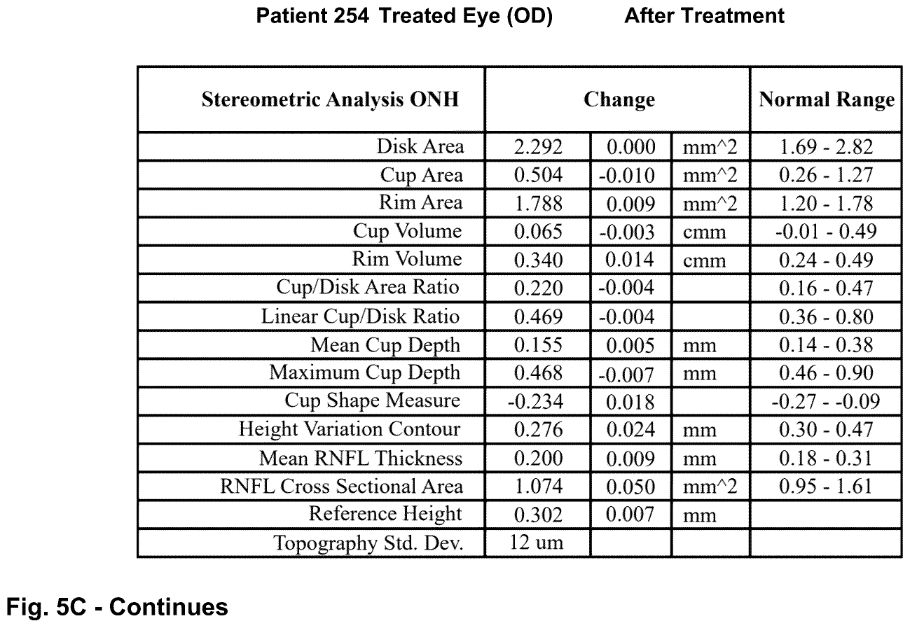

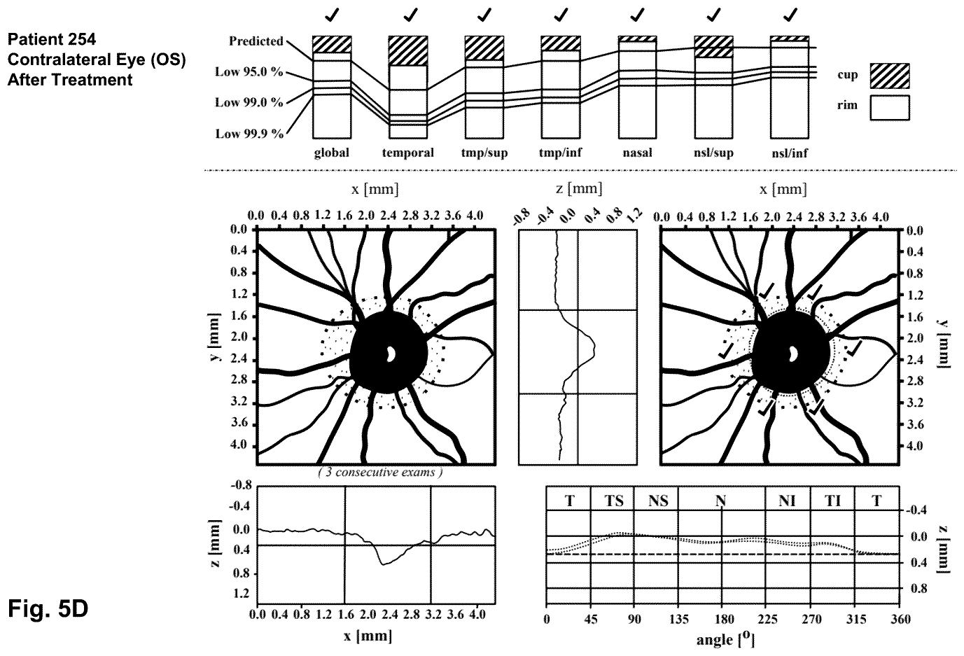

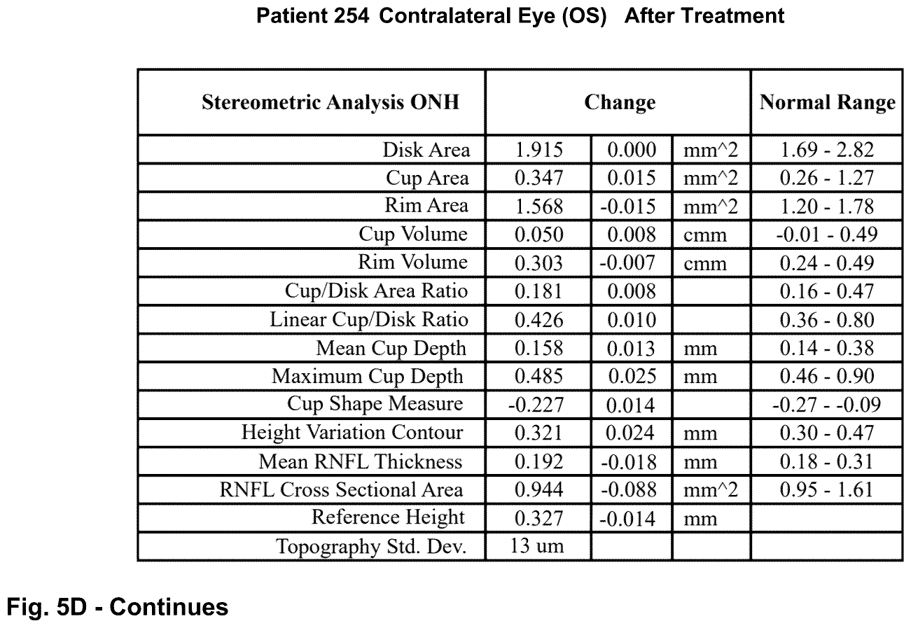

FIGS. 5A-D illustrate RNFL thickness (by measuring the thickness ganglion cell layer through the circumference adjacent to the optic nerve) and cup to disc ratio measured at baseline and follow-up visit of Patient 254. At baseline, the mean RNFL thickness was 0.182 mm and the cup to disc ratio was 0.23 mm in the treated eye (FIG. 5A). The mean RNFL thickness was 0.188 mm and the cup to disc ratio was 0.179 mm in the contralateral eye (FIG. 5B). At follow-up visit, the mean RNFL thickness was 0.2 mm and the cup to disc ratio was 0.22 mm in the treated eye (FIG. 5C). The mean RNFL thickness was 0.192 mm and the cup to disc ratio was 0.181 mm in the contralateral eye (FIG. 5D). Patient 254 had increased RNFL thickness by about 16.47% as compared to baseline thickness (Table 4).

The data demonstrate that ultrasound treatment is effective in decreasing the Cup/Disc ratio. The data showed that patients received ultrasound treatment had stable C/D ratio and many had a decrease in this parameter.

### 9.2 Example 2. Ultrasound Treatment Increased Vision Field

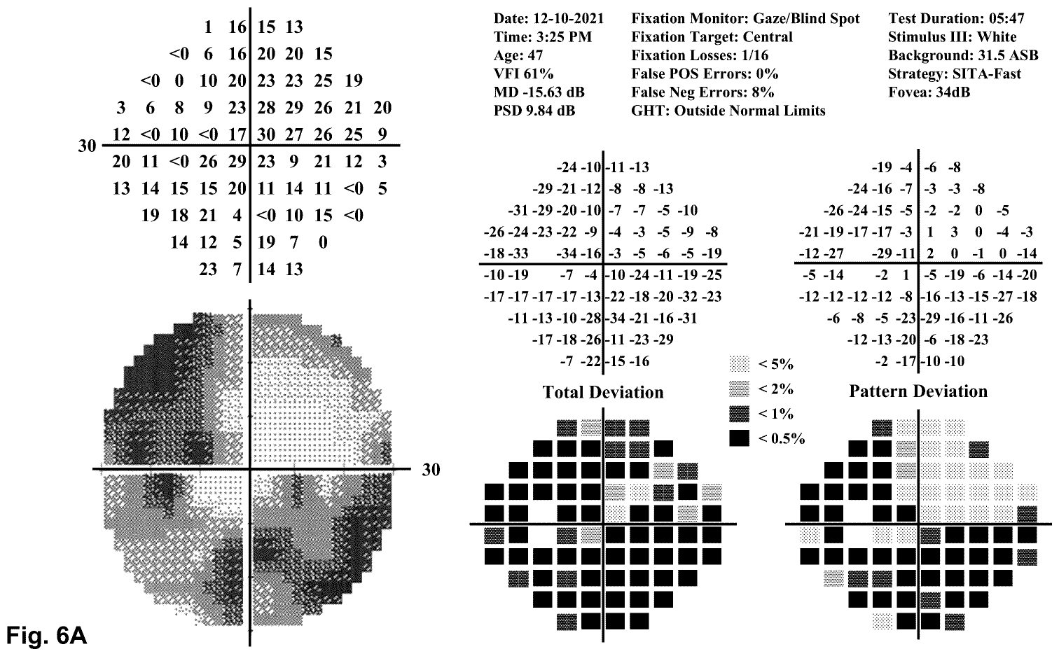

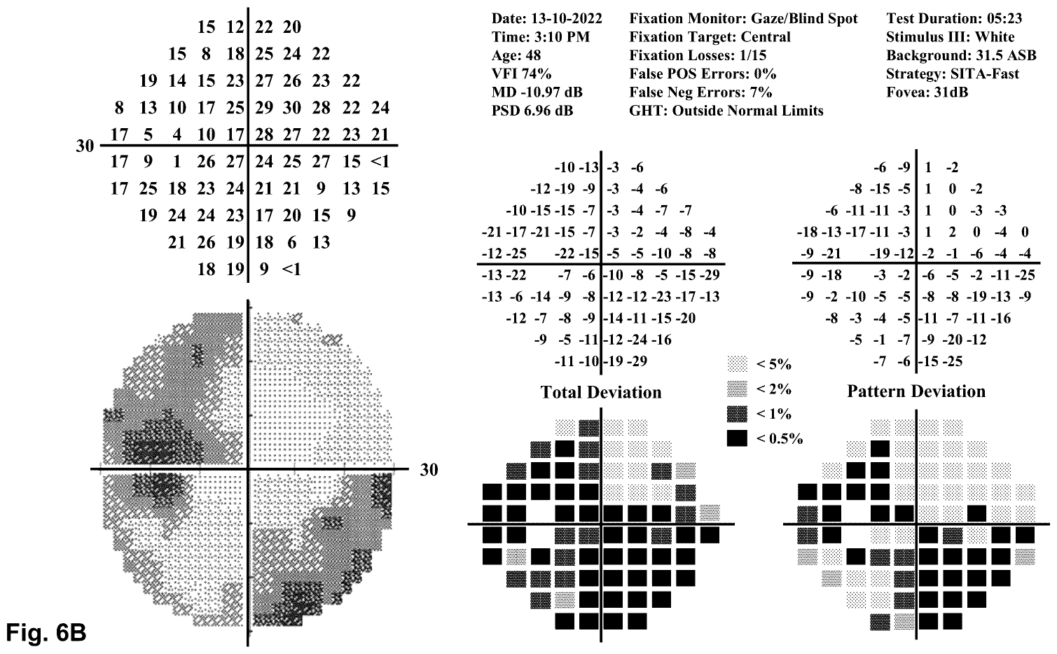

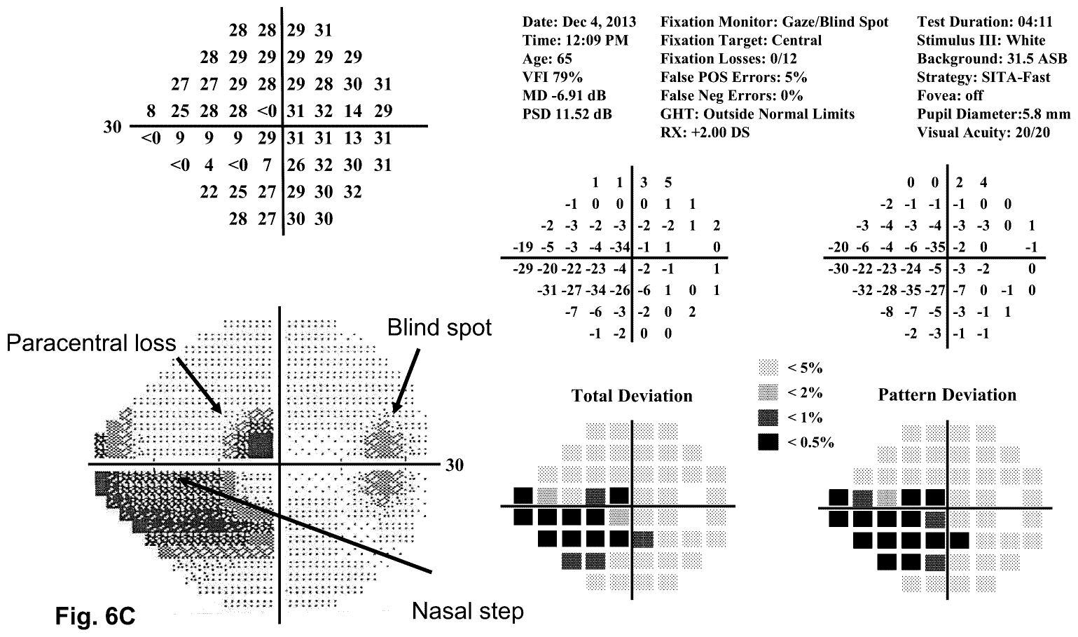

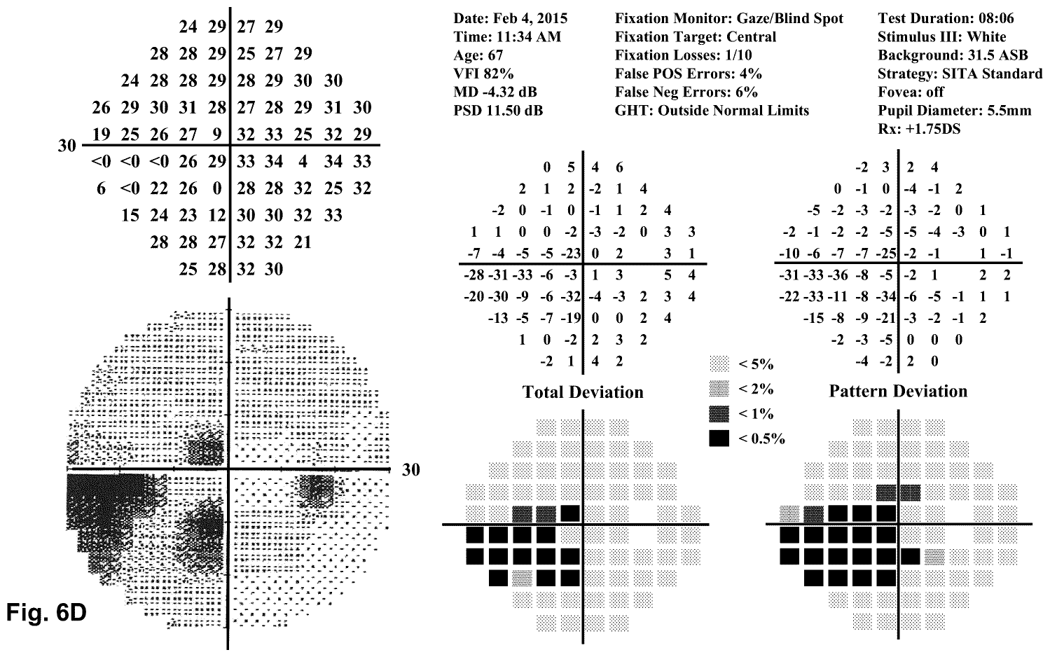

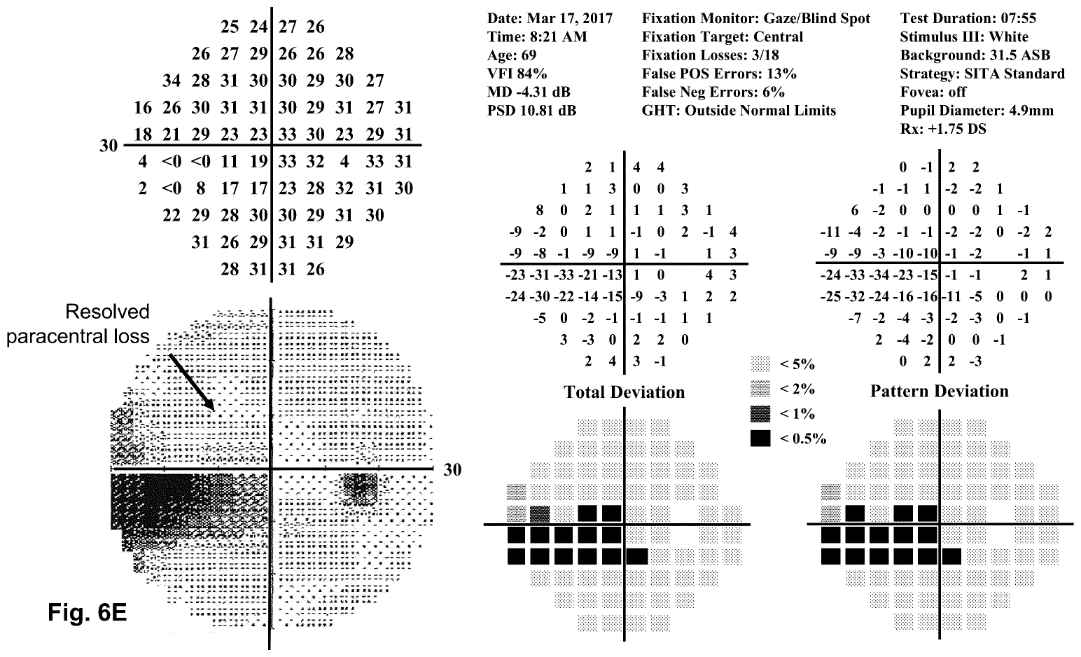

In a clinical investigation, four patients had their vision field (VFI) measured at baseline and 12 months after ultrasound treatment. Table 4 summarizes vision field (VFI) of 4 patients at baseline and 12 months. In general, ultrasound treatment increased VFI by about between 1.08% to about 66.67%. FIGS. 6A-6B illustrates VFI change in a representative patient from 61% at baseline (FIG. 6A) to 74% at 12 months (FIG. 6B).

Patient 262 had undiagnosed open angle glaucoma and was treatment naïve at the time of the Clinical Study I. Patient 262 had an IOP of 40 mmHg in the right eye (R) and 34 mmHg in the left (L) eye. There was evidence of significant optic nerve damage and an early vision field loss, ultrasound treatment was administered to the right eye of Patient 262. The treatment reduced the IOP from 40 mmHg to 24 mmHg, a decrease of 40% as compared to baseline.

Patient 262 had RNFL thickness of 62 μm at the baseline, which is significantly lower than the average range RNFL thickness: 102.37±7.45 (see e.g., Ocansey et al. Normative values of retinal nerve fiber layer thickness and optic nerve head parameters and their association with visual function in an African population. Vol 2020. Article ID 7150673). At 8 months post-ultrasound treatment, while Patient 262's RNFL appeared to have maintained the same (62 micron thick prior to and after ultrasound treatment), it was increased by 4.2% as compared to the baseline thickness when the natural RNFL annual thinning (e.g., 3.8%) per year was considered (Table 4). Patient 262 was monitored for about 2 years before dropping out of the study and began using pharmaceutical agents including latanoprost and dorzolamide for two years. Patient 262's IOP was controlled at about 25 mmHg during this period. By then, Patient 262 had two vision fields showing a large blind spot, a significant nasal step and a paracentral loss, which indicated severe glaucoma with a risk of losing central vision besides the peripheral vision loss. FIG. 6C shows Patient 262's VFI was about 79%, and a mean deviation of vision loss (MD) of −6.91 dB approximately two years after being off on ultrasound treatment and being placed on medications.

Patient 262 resumed receiving ultrasound treatment ancillary to the medications. The TOP of the right eye (treated) decreased from 24 mmHg to 16 mmHg, a reduction of about 33% as compared to medications alone. FIG. 6D shows Patient 262's VFI was about 82% at the baseline of the ancillary treatment and had a MD of −4.32 dB. FIG. 6E shows Patient 262's VFI was about 84% and an MD of −4.31 dB, after receiving ultrasound treatment for about 19 months. Ultrasound treatment improved the vision fields as indicated by the decreased size of the blind spot, nasal step, and recovery of paracentral area (arrow in FIG. 6E).

The data demonstrated that ultrasound treatment was effective in increasing VFI and was sustained over at least 12 months. Table 6. Summary of vision field index (VFI) of representative patients treated with ultrasound treatment.

TABLE 6
Summary of vision field index (VFI) of patients treated with ultrasound treatment

| | | | | | | | Days from | Changes from Baseline to | | |
|---|---|---|---|---|---|---|---|---|---|---|
| | Baseline | | | Follow-up | | | Baseline to | Follow-up | | |
| Patient | VFI | MD | PSD | VFI | MD | PSD | Follow-up | VFI | MD | PSD |
| 1 | 33 | −21.12 | 5.59 | 55 | −17.25 | 10.2 | 160 | 66.67% | −18.32% | 82.47% |
| 2 | 93 | −8.89 | 6.31 | 94 | −6.29 | 3.27 | 366 | 1.08% | −29.25% | −48.18% |
| 3 | 61 | −15.63 | 9.84 | 74 | −10.97 | 6.96 | 366 | 21.31% | −29.81% | −29.27% |
| 4 | 44 | −21.59 | 11.49 | 66 | −11.82 | 12.98 | 363 | 50.00% | −45.25% | 12.97% |

### 9.3 Example 3. Ultrasound Treatment Increased Tumor Necrosis Factor α (TNF-α) in Trabecular Meshwork

This example demonstrates ultrasound treatment increased Tumor Necrosis Factor alpha (TNF alpha) in trabecular meshwork. Tumor necrosis factor α (TNF-α) or TNF alpha is a secreted proinflammatory cytokine. Studies have shown that TNF alpha stimulated mesenchymal stem cells (MSCs) derived exosome can reduce retinal ganglion cell apoptosis induced by retinal ischemia-reperfusion injury (IRI), one of the main pathogenic mechanisms of glaucoma. See e.g., Ziyu et al. TNF-α stimulation enhances the neuroprotective effects of gingival MSCs derived exosomes in retinal ischemia-reperfusion injury via the MEG3/miR-21a-5p axis, Biomaterials, Volume 284, 2022, 121484. We hypothesized that TNF alpha could be synthesized by the trabecular meshwork in response to elevated IOP, acting to bring the pressure down and to protect optic nerve damage.

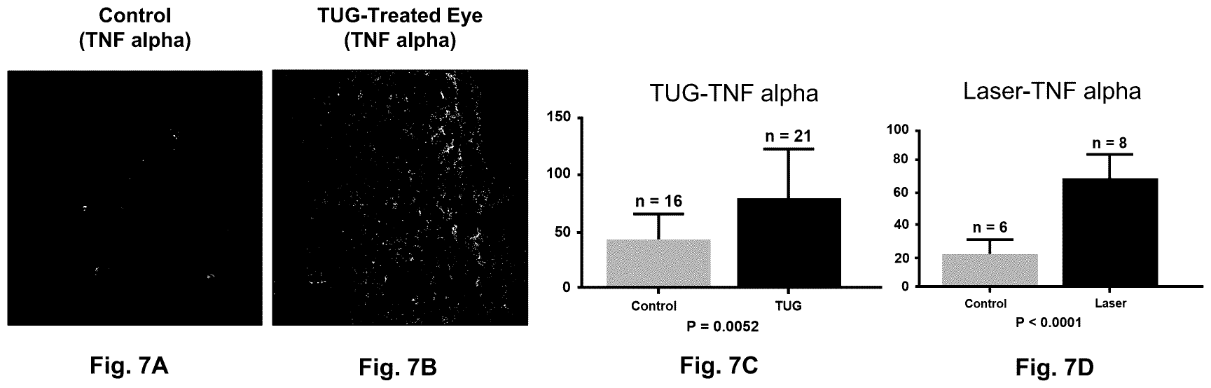

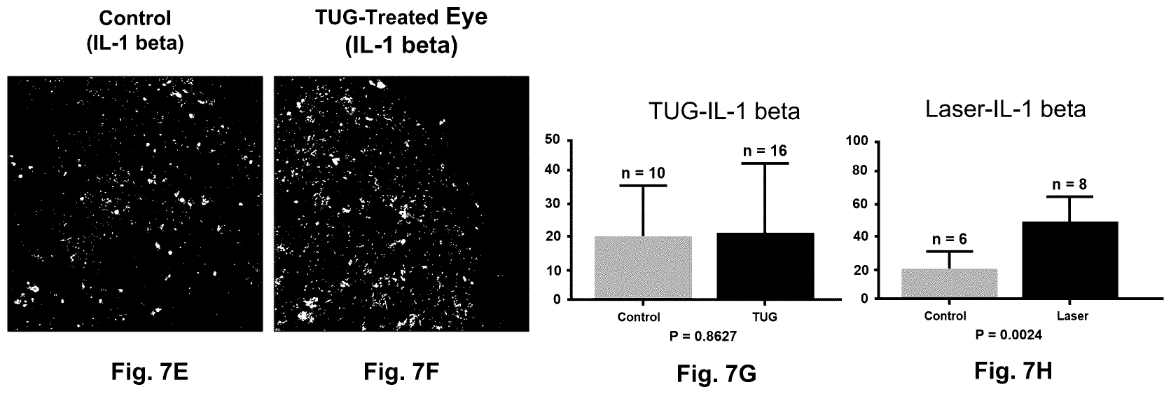

FIGS. 7A and 7B show anti-TNF alpha antibody staining in trabecular meshwork in the treated porcine eye as compared to the control porcine eye. FIG. 7C shows ultrasound treatment significantly (p=0.0052) increased TNF alpha in trabecular meshwork in the treated eye as compared to the sham control eye by ELISA. FIG. 7D shows laser treatment increased TNF alpha at a slightly lower level than ultrasound treatment.

FIGS. 7E and 7F show IL-1 beta antibody staining in trabecular meshwork in the treated porcine eye as compared to the control. FIG. 7G shows ultrasound treatment did not change IL-1 beta expression. FIG. 7H shows laser treatment increased IL-1 beta expression.

The data demonstrate that ultrasound treatment was effective in stimulating biomarkers for preventing optic nerve damage and/or promoting nerve repair or growth.

### 9.4 Example 4. Evaluation Ultrasound Treatment on Biomarker Expression in an Ex Vivo Model

This example evaluated the potential for a low power, low frequency non-invasive ultrasound to offer neuroprotection by biochemical analysis of bovine tissue treated with ultrasound treatment. Bovine eyes were used for this assay.

Bovine eyes were harvested and transported on ice to the treatment site within hours. Eyes were selected randomly for sham or treatment and kept on ice. The external ocular tissue was removed from the globes. Eyes were divided into two groups: (1) ultrasound treatment, and (2) sham control.

In the ultrasound treatment group, each eye was coated with Optixcare Eye Lube to serve as the contact lubricant for the ultrasound. Each eye received 12 applications of ultrasonic by contacting the ultrasonic device tip next to the limbus, over the ciliary body and aiming towards the pupil center. Each application lasted for 45s and delivered ultrasound at a frequency of 40 KHz. The power at the treatment tip of the transducer was maintained at about 45° C. In the sham control group, each eye was treated in the same manner except the ultrasonic device was turned off.

After the treatment, the treated and sham control eyes were incubated in MACS Buffer solution (auto MACS Running Buffer solution) at room temperature for 24 hours. Eyes were then dissected to collect tissue samples from the eye, including aqueous humor, trabecular meshwork, ciliary body and retina. Collected tissues were frozen at 80° C. and shipped on dry ice for biochemical analysis.

Expression level of biomarkers mTOR, GDNF, TNF, NGF, BDNF, and beta actin in the tissues were analyzed by Western Blot.

Tissues were dissected with clean tools, on ice preferably, and as quickly as possible to prevent degradation by proteases. The tissue was placed in round-bottom microcentrifuge tubes or Eppendorf tubes and immersed in liquid nitrogen to snap freeze. Samples were stored at −80° C. for later use or keep on ice for immediate homogenization. For an approximately 5 mg piece of tissue, about 300 μL of ice-cold lysis buffer was added rapidly to the tube, homogenized with an Qiagen TissueLyser. The homogenized tissue was centrifuged for 5-10 min at 14,000-17,000 g at 4° C. in a microcentrifuge. The supernatant was collected, and the protein contraction was determined.

Equal amounts of protein were loaded into the wells of a SDS-PAGE gel, along with a molecular weight marker. About 20-30 μg of total protein from cell lysate or tissue homogenate, or 10-100 ng of purified protein was loaded into each well. The gel was set to run 1-2 h at 100 V.

The gel was transferred to a nitrocellulose or PVDF membrane. The membrane was blocked for 1 h at room temperature or overnight at 4° C. using blocking buffer. Then, the membrane was incubated with appropriate dilutions of primary antibody in blocking buffer overnight at 4° C. The membrane was washed with TBST. Next, the membrane was incubated with the recommended dilution of IRDye 800 CW conjugated secondary antibody in blocking buffer at room temperature for 1 h, and was washed with TBST. Images were acquired using LiCor Odessey imager and analyzed by densitometry.

Studies in animal models have shown that mTOR inhibition prevents neurodegeneration. Rapamycin-induced inhibition of mTOR prevented glaucomatous neurodegeneration in a mice model of glaucoma (see e.g., Harder et al. Disturbed glucose and pyruvate metabolism in glaucoma with neuroprotection by pyruvate or rapamycin. PNAS. 117(52) 33619-33627). Rapamycin reduced IOP, promoted retinal ganglion cell survival, and increased retinal thickness in a microbead-induced chronic glaucoma rat model (see e.g., Wang et al. Topical administration of rapamycin promotes retinal ganglion cell survival and reduces intraocular pressure in a rat glaucoma model. Eur J Pharmacol. 2020 Oct. 5; 884:173369. doi: 10.1016/j.cjpbar.2020.173369). In a mouse model of retinal degeneration (retinitis pigmentosa), rapamycin improved retinal photoreceptor thickness and morphology, and could activate autophagy to remove the waste in the process of retinal photoreceptor cell death (see e.g., Zhao et al. Rapamycin improved retinal function and morphology in a mouse model of retinal degeneration. Front. Neurosci. Vol. 16. 2022. doi.org/10.3389/fnins.2022.846584).

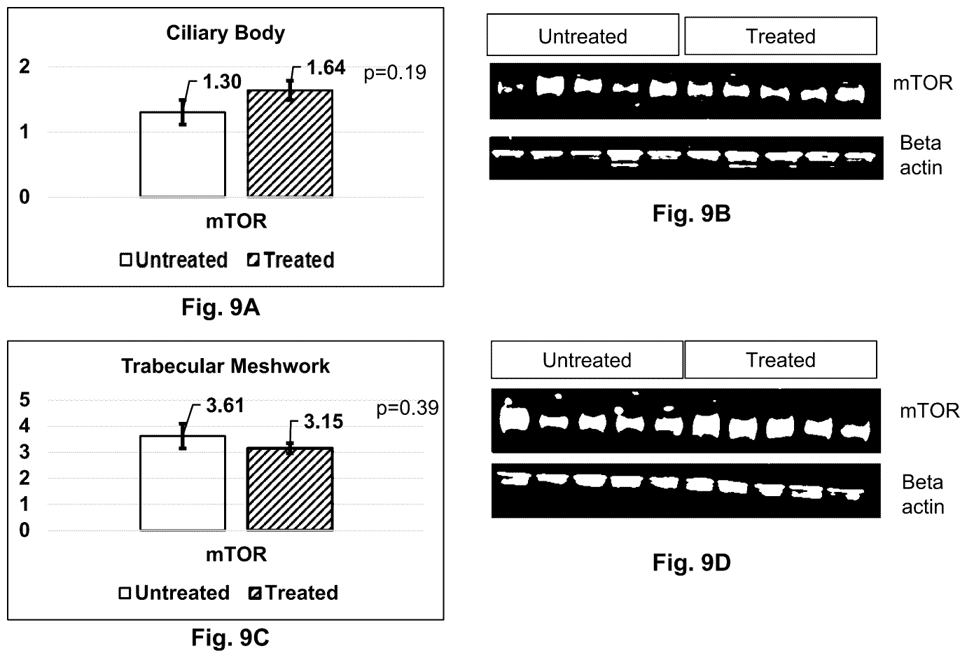

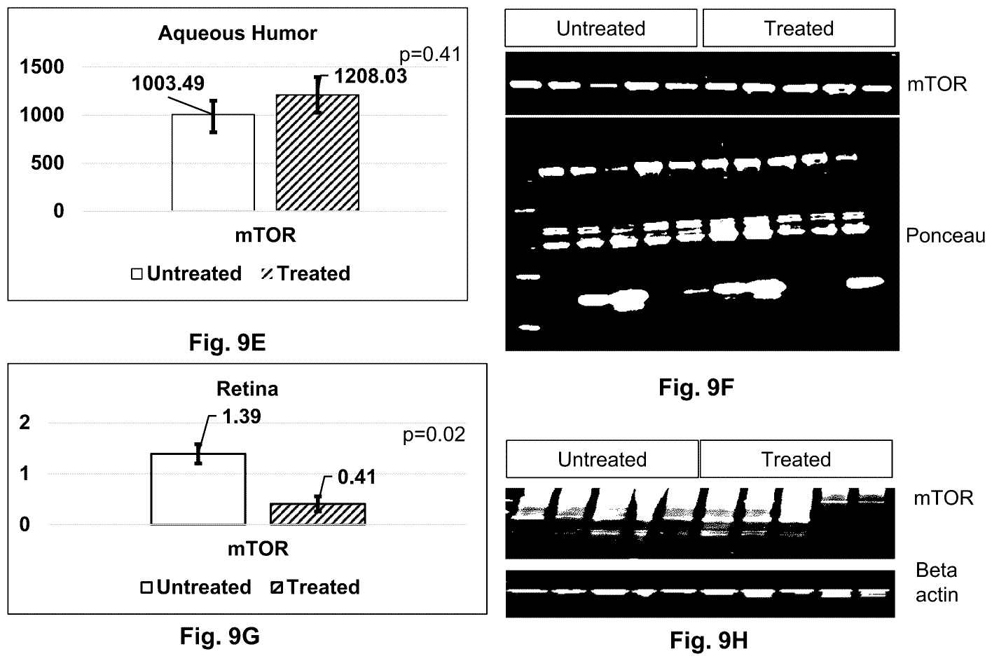

FIGS. 9A-9H provide data showing that a bovine eye treated with ultrasound had increased biomarkers expression in the ciliary body. Expression of beta actin was used as internal controls. FIGS. 9A and 9B show a greater presence of mTOR in the ciliary body of the bovine eye treated with ultrasound compared to the untreated eye. FIGS. 9C and 9D show no significant difference of mTOR in the trabecular meshwork in the eye with or without ultrasound treatment. FIGS. 9E and 9F show increased mTOR in aqueous humor after ultrasound treatment. In replacement for the lack of beta actin expression in the aqueous humor, the Ponceau imaged showed protein loading into each well. FIGS. 9G and 9H show significant reduction (p=0.02) of mTOR in the retina after ultrasound treatment. The data show that ultrasound treatment could reduce mTOR activity. Without being bound by a theory, ultrasound treatment-induced inhibition of mTOR in the trabecular meshwork can trigger activation of autophagy, leading to clearance of cellular debris and promoting survival. The reduction of mTOR in response to ultrasound treatment prevents neurodegeneration in the retina, leading to cell survival and providing neuroprotection.

The data demonstrate that ultrasound treatment is effective in preventing RNFL thinning and increasing RNFL thickness.

### 9.5 Example 5. Clinical Trial

This is a 12-month study with 6 months interim analysis endpoints and an additional 6 months of follow up in approximately 75 subjects with open angle glaucoma or ocular hypertension. The study involves a total of 8 visits. The first is a pre-enrollment screening visit with intraocular pressure readings recorded, a treatment visit, one day, 1 week, 1 month, 3 months, 6 months, and 12 months after treatment.

Glaucoma is a progressive optic neuropathy and a leading cause of blindness in adults over 60 years. According to the National Eye Institute, over 120,000 Americans are blind due to glaucoma. Elevated intraocular pressure (IOP) is an important risk factor for the development of glaucoma and is a result of abnormally high resistance to aqueous humor drainage through the trabecular meshwork, a multi-laminar array of collagen beams covered by endothelial-like cells.

The currently available glaucoma treatments all seek to decrease the IOP. Current treatments work on the mechanisms of action consisting of either decreasing aqueous fluid production in the ciliary epithelium or enhancing aqueous outflow via the trabecular meshwork (responsible for 80% of normal outflow) or the uveoscleral route (responsible for 20% of normal outflow), or a combination of these methods.

Pharmaceutical treatments include β-blockers, carbonic anhydrase inhibitors, prostaglandin analogs, α2-adrenergic agonists, and parasympathomimetic drugs. Argon laser trabeculoplasty was used for patients who were non-responsive to the pharmaceutical therapy. This method produced results; however late failure was commonly reported due to destruction of the trabecular meshwork with surrounding thermal damage. Cell rupture releases enzymes and other substances that trigger an inflammatory response. It is hypothesized that ultrasound treatment can trigger cytokines which result in the production of matrix metalloproteinase-3 and an induction of macrophages. This leads to a breakdown and removal of trabecular meshwork debris thereby decreasing resistance to outflow of aqueous and decreasing intraocular pressure.

The ultrasonic device is a non-invasive, low intensity, low frequency ultrasound. It is applied externally to the limbal area of the eye. The procedure is well tolerated and causes only minimal discomfort, typically including mild irritation and a slit-lamp finding of mild to moderate transient conjunctival hyperemia. The current study is aimed at using the ultrasonic device for IOP lowering effects, to be used in the treatment of Primary Open Angle Glaucoma. It is aimed at determining whether the treatment has a significant effect to decrease intraocular pressure.

This clinical trial is conducted in compliance with the protocol, International Conference on Harmonization (ICH), Good Clinical Practices (GCP) guidelines, Code of Federal Regulations Title 21 and other applicable guidelines and regulatory requirements.

Table 7 summarizes patient population and inclusion/exclusion criteria of the clinical trial.

TABLE 7
Clinical trial

| | |
|---|---|
| Objective | To assess the safety and efficacy of ultrasound treatment in |
| | subjects with primary open angle glaucoma |
| Overall study | This is a 2 arm, 52-week study in 75 subjects with primary |
| design | open angle glaucoma or Ocular Hypertension divided into |
| | three groups: naïve; monotherapy (one medication); |
| | multitherapy (more than one medication). |
| | This study has a total of 8 visits including a screening visit, a |
| | treatment visit, and 6 follow-up visits. The follow-up visits |
| | (post procedure) include: Day 1, 1 week, 1 month, 3 months, |
| | 6 months, and 12 months. |
| Study | Number of subjects: |
| population | Up to 75 subjects are planned to be enrolled to this study |
| | Study population characteristics: |
| | Subjects recruited can be male or female, of any race or |
| | ethnicity, at least 18 years of age at Visit 1, and present with |
| | Primary Open Angle Glaucoma or Ocular Hypertension with |
| | an intraocular pressure greater than 22 mmHg. All subjects |
| | enrolled must meet all the inclusion criteria and none of the |
| | exclusion criteria |
| Inclusion | To be enrolled in the study each subject must: |
| criteria | 1. Be at least 18 years of age at Visit 1, of either sex and any |
| | race or ethnicity |
| | 2. Be willing and able to provide written informed consent |
| | and Health Insurance Portability and Accountability Act |
| | (HIPAA) prior to any study procedures being performed. |
| | 3. Be willing and able to follow all instructions and attend all |
| | study visits. |
| | 4. Be willing to discontinue use of disallowed medication |
| | (see exclusion 14-17). |
| | 5. Have a best spectacle corrected visual acuity (BSCVA) of |
| | 0.60 logMAR or better in each eye as measured using an |
| | EDTRS chart at Visit 1. |
| | 6. Have a documented diagnosis of Primary Open Angle |
| | Glaucoma including Pigmentary Glaucoma or Ocular |
| | Hypertension with an intraocular pressure greater than 22 |
| | mmHg. |
| | 7. Pretreatment IOP values will be calculated as the average |
| | of up to three preceding visits to reduce fluctuations. Patients |
| | will be included if IOP ≥22 mmHg and ≤35 mmHg in the |
| | study eye. |
| Exclusion | 1. Single eye. |
| criteria | 2. Have any form of glaucoma other than primary open- |
| | angle glaucoma or pigmentary glaucoma. |
| | 3. History of ocular infection (bacterial, viral or fungal), |
| | or ocular inflammation within the past 3 months in either eye. |
| | 4. History of chronic or recurrent severe inflammatory |
| | eye disease (i.e., scleritis, uveitis, iritis, herpes keratitis) in |
| | either eye. |
| | 5. Any corneal abnormality preventing reliable |
| | applanation tonometry in either eye. |
| | 6. Nystagmus. |
| | 7. History of any glaucoma-related procedure whether |
| | laser or incisional in either eye (trabeculectomy, laser |
| | iridotomy, laser trabeculoplasty, etc.) or planned during the |
| | course of the study. |
| | 8. Have had any ocular surgery (e.g., intraocular laser |
| | surgery, cataract surgery or eyelid surgery), or ocular trauma |
| | in treatment eye. |
| | 9. Had had any corneal refractive laser surgery (laser- |
| | assisted in-situ keratomileusis [LASIK], photorefractive |
| | keratectomy [PRK], radial keratotomy [RK]) in either eye or |
| | planned during the course of the study. |
| | 10. Have an IOP in either eye at any time point during the |
| | Screening or Baseline visits (Visits 1 or 2) of >35 mmHg. |
| | 11. Have a central corneal thickness greater than 600 μm or |
| | less than 500 μm in the study eye |
| | 12. Cup to disc ratio >0.80 (horizontal or vertical |
| | measurement) in treatment eye. |
| | 13. Clinically relevant severe central vision field defect or |
| | documented significant progression of a vision field defect |
| | during the last year in treatment eye, which in the opinion of |
| | the investigator may place the subject at risk to participate in |
| | the study. |
| | 14. History of retinal, macular or optic nerve disease apart |
| | from glaucoma. |
| | 15. Have used steroid topical ophthalmic solutions on the |
| | same day as Visit 1 and for the duration of the study. |
| | 16. Be on systemic medications that can have an impact on |
| | IOP unless such medications have been used for at least 3 |
| | months prior to study enrollment (Visit 2) and are likely to |
| | remain constant throughout the duration of the study. |
| | 17. Have prior (within 30 days of Visit 1) or anticipated |
| | concurrent use of an investigational drug or device. |
| | 18. Have a condition (ocular or systemic) or a situation |
| | which, in the Investigator's opinion, may put the subject at |
| | increased risk, may confound study data, or may interfere |
| | significantly with the subject's study participation. |
| Study eye | One eye will be treated with ultrasound treatment. If at any |
| selection | time during the study the investigator deems that the subject's |
| | safety has been compromised the subject may be withdrawn |
| | from the study. If there is an increase in intraocular pressure |
| | after the 12-week visit, the subject may be offered by |
| | physician an additional ultrasound treatment, pending |
| | physician consideration. |
| | If at any time during the study an enrolled subject has an IOP |
| | increase of greater than 10 mmHg from baseline in either eye, |
| | the investigator should withdraw the subject from the study |
| | and prescribe IOP lowering medication(s) or repeat the |
| | ultrasound treatment. The investigator should follow the |
| | subject to monitor the IOP until the investigator determines it |
| | is properly controlled |
| | Any subject who wishes to withdraw from the study for any |
| | reason is entitled to do so at any time without obligation. The |
| | reason(s) for discontinuation of treatment must be recorded in |
| | the subject's source document. |
| Endpoints | Primary |
| | Safety |
| | SAE to be monitored: death, loss of vision. |
| | AE to be monitored: Inflammation with the |
| | occurrence of greater than +3 cells, change in |
| | vision, increased discomfort, or pain. Increased |
| | intraocular pressure of ≥20%. |
| | Efficacy: IOP ≥20% reduction from baseline. |
| | Secondary |
| | Efficacy |
| | Percentage of patients with medication |
| | reduction following treatment. |
| | Visual Field at 6 Months and 12 Months. |
| | RNFL thickness layer increase ≥5% as seen in |
| | OCT at 12 Months |

TABLE 8
summarizes clinical evaluations and study calendar.
Clinical evaluations and study calendar

| | | Ultrasound | 1 | 1 | 1 | 3 | 6 | 12 |
|---|---|---|---|---|---|---|---|---|
| | Evaluation | treatment | Day | Week | Month | Month | Month | Month |
| Visual Acuity | X | X | X | X | X | X | X | X |
| History & Physical | X | | | | | | | |
| Examination | | | | | | | | |
| Slit Lamp | X | X | X | X | X | X | X | X |
| Intraocular Pressure | X | X | X | X | X | X | X | X |
| Optical Coherence | X | | | | | | X | X |
| Tomography | | | | | | | | |
| Ultrasound Treatment | | X | | | | | X (Optional) | |
| Vision Field | X | | | | | | X | X |
| Contrast | X | | | | | | | X |
| Sensitivity | | | | | | | | |

The data demonstrate that ultrasound treatment decreases intraocular pressure and allows a patient on glaucoma treatment to decrease their present glaucoma treatment regimen or eliminate the need for pharmaceutical control. The data demonstrate that ultrasound treatment provides neuroprotection of the optic nerve and increases RNFL thickness, and/or prevents RNFL thinning due to glaucoma or elevated IOP. Ultrasound thus can be used to supplement and replace first-line pharmaceutical treatments of glaucoma, circumventing compliance issues and reducing reliance on invasive surgical treatments.

### 9.6 Example 6. Ultrasound Treatment Stimulates Gene Expression in Retinal Ganglion Cells

This example is designed to demonstrate that ultrasound treatment stimulates gene expression in retinal ganglion cells.

This study is conducted in accordance with the Association for Research in Vision and Ophthalmology (ARVO) Statement for the Use of Animals in Ophthalmic and Vision Research. Groups of animals are treated with ultrasound: Group 1 (control non-treated); Group 2 (treated and 6 hr); Group 3 (treated and 12 hr); Group 4 (treated and 24 hr); Group 5 (treated and 48 hr). Before ultrasound treatment, animals are anesthetized with intraperitoneal pentobarbital (50 mg/kg) injection and pupils are dilated with tropicamide and phenylephrine eye drops (0.5% each). Animals are given ultrasound treatment as described herein. Animals are sacrificed at the designated time points. Control, treated and contralateral eyeballs are collected for RNA and protein extraction.

Eyeballs are enucleated 0 hr (control) 6 hr, 12 hr, 24 hr, and 48 hr after ultrasound treatment after pentobarbital overdose. Enucleated eyeballs are immersed in cold Hanks' balanced salt solution immediately after enucleation. Incisions are made using pinholes in the corneas, then using the incisions, the sclera is peeled to remove the mixture of the retinal pigment epithelium, choroid and sclera from the neural retina as previously described (Hasegawa. et al. Sci. Rep. 6, 31184 (2016), which incorporated herein by reference in its entirety). The lens and iris are removed. RNA and protein are isolated from the neural retina using the AllPrep RNA/Protein Kit (QIAGEN). The mRNA was reverse transcribed with the M-MLV reverse transcriptase (Promega, WI, USA) and then complementary DNA was amplified by PCR with SYBR premix Ex Taq polymerase (Takara Bio Inc., Shiga, Japan) and 60 ° C. as the annealing temperature on the 7300 Real-Time PCR System (Applied Biosystems, CA, USA).

mRNA profiling of biomarkers of interest such as mTOR, WNT, NGF, GDNF, BDNF, IL-1, IL-6, IL-8, TNF-alpha, Akt1, Pax6, Bcl-2, and Brn3a, is performed using quantitative PCR. Actin is used as a control. The ratios of mRNA expression of each gene to that of actin are calculated. The ratios of mRNA expression of each gene in Groups 2-5 are compared to Group 1 (control) using an unpaired t-test.

The results show ultrasound treatment modulates gene expression of biomarkers in at least one of the signaling pathways including mTOR, TNF, WNT, NGF, and GDNF as illustrated in FIG. 2. Ultrasound treatment increases or decreases mTOR, WNT, NGF, GDNF, BDNF, IL-1, IL-6, IL-8, TNF-alpha, Akt1, Pax6, Bcl-2, and Brn3a in retinal ganglion cells of treated eyes and to a lesser degree or comparable degree, in the contralateral eyes.

The data demonstrate that ultrasound treatment is effective in modulating retinal ganglion cell growth and increasing RNFL thickness.

### 9.7 Example 7. Ultrasound Treatment Stimulates Protein Expression in Retinal Ganglion Cells

This example is designed to demonstrate that ultrasound treatment stimulates protein expression in retinal ganglion cells.

Groups of the same animals as described in Example 6 are treated with ultrasound: Group 1 (control non-treated); Group 2 (treated and 6 hr); Group 3 (treated and 12 hr); Group 4 (treated and 24 hr); Group 5 (treated and 48 hr). Animals are given ultrasound treatment as described herein. Animals are sacrificed at the designated time points. Control, treated and contralateral eyeballs are collected for immunostaining and detection of antibodies of interest, including anti-IL-6, anti-TNF-alpha in the retinal ganglion cells.

Eyeballs are collected and processed as described in Example 6. Protein extraction from the same samples in Example 6 is performed using the suitable RNA and protein purification kits, e.g., AllPrep RNA/Protein Kit (QIAGEN).

Protein expression of biomarkers of interest such as mTOR, WNT, NGF, GDNF, BDNF, IL-1, IL-6, IL-8, TNF-alpha, Akt1, Pax6, Bcl-2, and Brn3a, is performed using Western blot analysis. Actin is used as a control.

The results show ultrasound treatment modulates protein expression of biomarkers in at least one of the signaling pathways including mTOR, TNF, WNT, NGF, and GDNF as illustrated in FIG. 2. Ultrasound treatment increases or decreases protein expression of mTOR, WNT, NGF, GDNF, BDNF, IL-1, IL-6, IL-8, TNF-alpha, Akt1, Pax6, Bcl-2, and Brn3a n retinal ganglion cells of treated eyes and to a lesser degree or comparable degree, in the contralateral eyes.

The data demonstrate that ultrasound treatment is effective in stimulating retinal ganglion cell growth and increasing RNFL thickness.

### 9.8 Example 8. Ultrasound Treatment Increases RNFL Thickness

This example is designed to demonstrate ultrasound treatment increases RNFL thickness.

Groups of animals are handled and treated as described in Example 6. Group 1 (control non-treated); Group 2 (treated and 1 week); Group 3 (treated and 2 weeks); Group 4 (treated and 3 weeks); Group 5 (treated and 4 weeks). Animals are given ultrasound treatment as described herein. Animals are sacrificed at the designated time points. Control, treated, and contralateral eyeballs are collected and handled as described in Example 6. Treated and contralateral eyeballs are prepared for immunostaining and detection of antibodies of interest, such as anti-IL-6, anti-TNF-alpha, anti-Pax6, and anti-Brn₃a in the RNFL. The expression of antibodies are quantified. The RNFL is measured and quantified.

The results show ultrasound treatment increases protein expression of biomarkers in at least one of the signaling pathways including mTOR, TNF, WNT, NGF, and GDNF as illustrated in FIG. 2. Ultrasound treatment increases protein expression of IL-1, IL-6, IL-8, TNF-alpha, Akt1, Pax6, Bcl-2, mTOR, and Brn3a in RNFL of treated eyes and to a lesser degree or comparable degree, in the contralateral eyes. Ultrasound treatment increases RNFL thickness in treated eyes and contralateral eyes.

The data demonstrate that ultrasound treatment is effective in stimulating RNFL cell growth and increasing RNFL thickness. Restoring or enhancing the optic nerve health is essential for treating and/or reducing progression of glaucoma. The data demonstrate that the use of ultrasound treatment not only decreased intraocular pressure, but also addressed the central cause of glaucoma visual loss by enhancing the health of the optic nerve and the retinal nerve fiber layer.

In conclusion, conventional glaucoma treatment is to reduce intraocular pressure. Here, the inventor discovered (1) ultrasound treatment (ultrasonic energy) can stimulate cell growth in the RNFL as measured via the thickness, (2) that stimulating cell growth in the RNFL can effectively reduce glaucoma symptoms, reduce the cup/disk ratio, and increase the vision field, (3) ultrasound treatment can be administered to the patient in one or more sessions to increase or maintain treatment effectiveness, (4) ultrasound treatmentas a monotherapy is effective in treating glaucoma and/or eye disorder related to RNFL thinning in patients who have not been treated with existing glaucoma treatments, or patients whose glaucoma progressed following traditional treatments; and (5) ultrasound treatmentis effective as an ancillary treatment to potentiate pharmaceutical agents to achieve additive treatment effects.

### 10. EMBODIMENTS

Embodiment 1. A method of improving vision in a subject suffering from or at risk of glaucoma or associated visual disorders, the method comprising:
providing ultrasonic energy using an ultrasonic device that emits ultrasonic energy to deliver between 0.01-3.99 watts of power to the trabecular meshwork of an eye,
wherein the energy is concentrated from between 0.01-10 mm from the tip of the sonotrode of the device,
wherein the ultrasonic energy propagates throughout the eye,
wherein the ultrasonic energy stimulates cell growth in the retina nerve fiber layer (RNFL), thereby improving vision of the subject.

Embodiment 2. A method of increasing thickness of retina nerve fiber layer (RNFL) in a subject suffering from a visual disorder, the method comprising:
providing ultrasonic energy using an ultrasonic device that emits ultrasonic energy to deliver between 0.01-3.99 watts of power to the trabecular meshwork of an eye,
wherein the energy is concentrated from between 0.01-10 mm from the tip of the sonotrode of the device,
wherein the ultrasonic energy propagates throughout the eye,
wherein the ultrasonic energy stimulates one or more biomarkers in one or more cell signaling pathways involved in promoting cell growth in the retina nerve fiber layer (RNFL), thereby increasing thickness of the RNFL.

Embodiment 3. The method of embodiment 2, wherein the one or more cell signaling pathways are selected from mTOR, TNF, WNT, NGF, and GDNF.

Embodiment 4. The method of embodiment 2 or embodiment 3, wherein the one or more biomarkers are selected from IL6, TNF-alpha, Akt1, Pax6, Bcl-2, and Brn3a in the RNFL.

Embodiment 5. The method of embodiment 2, wherein the ultrasonic energy increases expression level of biomarkers selected from neurotrophin 3, CNTF, HSP70, or any combination thereof.

Embodiment 6. The method of any one of embodiments 1-5, wherein the ultrasonic energy increases gene expression level of IL6, TNF-alpha, Akt1, Pax6, Bcl-2, and Brn3a in retinal ganglion cells (RGC).

Embodiment 7. The method of any one of embodiments 1-6, wherein the ultrasonic energy increases protein expression level of IL6, TNF-alpha, Akt1, Pax6, Bcl-2, and Brn3a in retinal ganglion cells (RGC)

Embodiment 8. The method of any one of embodiments 1-7, wherein the ultrasonic energy increases axonal growth in the RNFL.

Embodiment 9. The method of any one of embodiments 1-8, further comprising adjusting the temperature of a portion of the eye to a temperature of between about 41 and about 45 degrees Centigrade to initiate a biochemical cascade within the eye.

Embodiment 10. The method of any one of embodiments 1-9, wherein the movement of the sonotrode tip is between 1-10 μm.

Embodiment 11. The method of any one of embodiments 1-10, wherein the ultrasonic device delivers between about 0.5-2.5 watts of power to the trabecular meshwork of the eye.

Embodiment 12. The method of embodiment 10, wherein the ultrasonic device delivers about 2 watts of power to the trabecular meshwork of the eye.

Embodiment 13. The method of any one of embodiments 1-11, wherein the ultrasonic energy is applied at a frequency range of about 10,000 Hz to about 100,000 Hz.

Embodiment 14. The method of embodiment 13, wherein the frequency is between about 40,000 Hz to about 65,000 Hz.

Embodiment 15. The method of any one of embodiments 1-14, wherein the time is between about 5 seconds and about 120 seconds.

Embodiment 16. The method of embodiment 15, wherein the time is about 45 seconds.

Embodiment 17. The method of any one of embodiments 1-15, wherein the energy is concentrated from between 0.01-2.00 mm from the tip of the sonotrode of the device.

Embodiment 18. The method of any one of embodiments 1-17 wherein the subject has glaucoma.

Embodiment 19. The method of any one of embodiments 1-17, wherein the subject has optic neuropathies.

Embodiment 20. A device for delivering ultrasonic energy to a subject for improving vision of any one of embodiments 1 and 6-19, or increasing thickness of the RNFL of any one of embodiments 2-19.

Embodiment 21. A device for performing the method of embodiment 1 for improving vision in a subject suffering from or at risk of glaucoma or associated visual disorders.

Embodiment 22. The device of embodiment 21, wherein the device is as described in FIGS. 8A-8C.

Embodiment 23. A device for preforming the method of embodiment 2 for increasing thickness of retina nerve fiber layer (RNFL) in a subject suffering from a visual disorder.

Embodiment 24. The device of embodiment 23, wherein the device is as described in FIGS. 8A-8C.

### 11. EQUIVALENT AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes. The references include, but not limited to Schwartz et al . . . . Therapeutic ultrasound for glaucoma: clinical use of a low-frequency low-power ultrasonic device for lowering intraocular pressure. J Ther Ultrasound. 2014 Sep. 26; 2:15; U.S. Pat. Nos. 7,909,781; 8,043,235; and 9,125,722; registered clinical trial ISRCTN50904302, each of which is hereby incorporated by reference in its entirety.

What is claimed is:

1. A method of treating a subject suffering from or at risk of an eye disorder, the method comprising:
providing ultrasonic energy to the limbus of an eye at an effective treatment schedule using an ultrasonic device that emits ultrasonic energy of between 0.01-5 watts of power,
wherein the ultrasonic energy is concentrated at a distance between 0.01-10 mm from the tip of the sonotrode of the ultrasonic device,
wherein the ultrasonic energy propagates throughout the eye,
wherein the effective treatment schedule is sufficient to stimulate cell growth in the retina nerve fiber layer (RNFL) in the subject.

2. The method of claim 1, wherein the effective treatment schedule is sufficient to increase or decrease the expression level of one or more biomarkers involved in promoting cell growth in the retina nerve fiber layer (RNFL), the one or more biomarkers are selected from the mammalian target of rapamycin (mTOR), wingless-related integration site (WNT), nerve growth factor (NGF), glial cell line-derived neurotrophic factor (GDNF), brain-derived neurotrophic factor (BDNF), interleukin 1 (IL-1), interleukin 6 (IL-6), interleukin 8 (IL-8), tumor necrosis factor-alpha (TNF-α), matrix metalloproteinase 3 (MMP-3), Ak strain transforming 1 (Akt1), paired box 6 (Pax6), B-cell leukemia/lymphoma 2 (Bcl-2), ciliary neurotrophic factor (CNTF), heat shock protein 70 (HSP70), and brain-specific homeobox/POU domain protein 3 (Brn3a) in the retina.

3. The method of claim 1, wherein the effective treatment schedule is sufficient to increase axonal growth in the RNFL or thickness of the RNFL by at least 1%, 2%, 3%, 4%, 5%, 10%, 15%, 20%, 30%, 40%, 50%, 100%, 150%, 200%, 300%, 400% or more as compared to baseline.

4. The method of claim 3, wherein the effective treatment schedule is sufficient to increase the RNFL thickness in both treated and untreated eyes.

5. The method of claim 4, wherein the RNFL thickness is measured by Heidelberg Retinal Tomography (HRT) or optical coherence tomography (OCT).

6. The method of claim 1, wherein the effective treatment schedule is sufficient to decrease a cup to disc ratio of the eye measured by Optical coherence tomography (OCT) or Heidelberg Retinal Tomography (HRT).

7. The method of claim 1, wherein the effective treatment schedule is sufficient to reduce an elevated intraocular pressure (IOP) in the subject's eye to the level of a healthy eye or lower, and/or increase vision field of the eye.

8. The method of claim 1, wherein the effective treatment schedule comprises two or more sessions of providing ultrasonic energy to the limbus of the eye.

9. The method of claim 8, wherein the two or more sessions are scheduled 1 month, 3 months, 6 months, or 12 months apart from each other; or the two or more sessions are scheduled over a period of every 1 month, every 3 months, every 6 months or every 12 months.

10. The method of claim 1, wherein the subject has glaucoma, is suspected of having glaucoma, has not been diagnosed with glaucoma, or is not suspected of having glaucoma.

11. The method of claim 1, wherein the subject has advanced visual disease, elevated intraocular pressure, does not have elevated intraocular pressure, diurnal intraocular pressure (IOP), old age, decreased central corneal thickness, disc hemorrhage, genetic mutations, large beta zone of peripapillary atrophy, or optic neuropathies.

12. The method of claim 11, wherein the subject has open-angle glaucoma (OAG) or ocular hypertension.

13. The method of claim 11, wherein the subject has normotensive glaucoma.

14. The method of claim 1, wherein the subject has retinal nerve fiber layer (RNFL) thinner than 80 μm, thinner than 70 μm, thinner than 60 μm, thinner than 50 μm, thinner than 40 μm or thinner than an average RNFL thickness in a healthy adult.

15. The method of claim 14, wherein the subject has anemia, multiple sclerosis, age-related RNFL loss, retinitis pigmentosa, anterior ischemic optic neuropathy, high myopic retinal degeneration, or macular degeneration.

16. The method of claim 1, wherein the subject has not received alternative glaucoma treatment before the ultrasonic treatment selected from intervention surgery and pharmaceutical treatments; or the subject has received one or more alternative glaucoma treatments before the ultrasonic treatment.

17. The method of claim 16, wherein the effective treatment schedule is sufficient to reduce need for the alternative glaucoma treatment.

18. The method of claim 1, further comprising adjusting the temperature of a portion of the eye to a temperature of between about 41 and about 45 degrees, about 40 and about 48 degrees, or about 35 and about 55 degrees Centigrade.

19. The method of claim 1, wherein the ultrasonic energy concentrated at the distance from the tip of the sonotrode of the ultrasonic device is less than 4 watts of power.

20. The method of claim 1, wherein the ultrasonic energy from the ultrasonic device has about 30-60 KHz frequency.

21. The method of claim 1, wherein the ultrasonic energy is applied as 10 to 15 applications wherein the 10 to 15 applications are applied while moving the tip of the sonotrode in one direction on the limbus.

22. The method of claim 21, wherein each of the applications lasts between 5 seconds and 120 seconds, or about 45 seconds.

23. The method of claim 1, wherein the ultrasonic intensity is concentrated at a distance between 0.01-2.00 mm, 0.01-10 mm, 0.5-5 mm, or 1-3 mm from the tip of the sonotrode of the ultrasonic device.

24. The method of claim 1, wherein the ultrasound energy is provided at 45 degrees with respect to a normal vector of a surface of the limbus.

25. The method of claim 1, wherein the ultrasound energy is divergent, low intensity, and low frequency ultrasonic energy.

26. The method of claim 1, wherein the ultrasonic energy is provided toward the trabecular meshwork of the subject's eye.

27. A method of increasing thickness of retina nerve fiber layer (RNFL) in a subject, the method comprising:
providing ultrasonic energy to the limbus of an eye at an effective treatment schedule using an ultrasonic device that emits ultrasonic energy of between 0.01-5 watts of power,
wherein the ultrasonic energy is concentrated at a distance between 0.01-10 mm from the tip of the sonotrode of the ultrasonic device,
wherein the ultrasonic energy propagates throughout the eye,
wherein the effective treatment schedule is sufficient to modulate one or more biomarkers involved in promoting cell growth in the retina nerve fiber layer (RNFL), thereby increasing thickness of the RNFL.

28. A device for use in a treatment method of treating a subject suffering from or at risk of an eye disorder, the method comprising:
providing ultrasonic energy to the limbus of an eye at an effective treatment schedule using an ultrasonic device that emits ultrasonic energy of between 0.01-5 watts of power,
wherein the ultrasonic energy is concentrated at a distance between 0.01-10 mm from the tip of the sonotrode of the ultrasonic device,
wherein the ultrasonic energy propagates throughout the eye,
wherein the effective treatment schedule is sufficient to stimulate cell growth in the retina nerve fiber layer (RNFL) in the subject.
